(19) United States
(12) Patent Application Publication
(10) Pub. No.: US 2023/0133936 A1
(43) Pub. Date: May 4, 2023

(54) NON-INVASIVE DETECTION METHOD, DEVICE, SYSTEM AND WEARABLE APPARATUS FOR TISSUE ELEMENT

(71) Applicant: SUNRISE TECHNOLOGIES CO., LTD., Beijing (CN)

(72) Inventor: Kexin Xu, Beijing (CN)

(21) Appl. No.: 17/802,460

(22) PCT Filed: Feb. 20, 2021
(86) PCT No.: PCT/CN2021/077071

§ 371 Date: Aug. 25, 2022

(30) Foreign Application Priority Data
Feb. 26, 2020 (CN) 202010120521.6

(51) Int. Cl. A61B 5/00; A61B 5/145

(52) U.S. Cl. CPC A61B 5/681; A61B 5/0075; A61B 5/14532; A61B 5/6824

(57) ABSTRACT

A non-invasive detection method, device, system and wearable apparatus for tissue element. The method includes: acquiring, for a detected site of a detected object, second light intensity measurement value for each predetermined wavelength of at least one predetermined wavelength at a measurement distance, and/or a second light intensity reference value for each predetermined wavelength of at least one predetermined wavelength at a reference distance, wherein the measurement distance is a source-detection distance corresponding to a first light intensity measurement value, and the reference distance is a source-detection distance corresponding to a first light intensity reference value and determining a concentration of a tissue element to be detected according to the second light intensity measurement value for each predetermined wavelength and/or the second light intensity reference value for each predetermined wavelength.

### CROSS REFERENCE TO RELATED APPLICATION(S)

This application is a Section 371 National Stage Application of International Application No. PCT/CN2021/077071, filed on Feb. 20, 2021, entitled “NON-INVASIVE DETECTION METHOD, DEVICE, SYSTEM AND WEARABLE APPARATUS FOR TISSUE ELEMENT”, which claims priority to Chinese Patent Application No. 202010120521.6 filed on Feb. 26, 2020, and the contents of which are incorporated herein by reference in their entirety.

### TECHNICAL FIELD

The present disclosure relates to a field of spectrum detection technology, and in particular, to a non-invasive detection method, device, system and wearable apparatus for a tissue element.

### BACKGROUND

A near-infrared spectroscopy detection method has characteristics of rapidness, non-invasiveness, and multidimensional information, etc., and is generally adopted to detect a tissue element, including blood glucose, fat, and white blood cells, etc. However, due to a weak absorption of the tissue element to be detected and a small change in a concentration of the tissue element to be detected of a detected object, a valid signal for detection is weak. Moreover, it is very vulnerable to an interference of a human body background and a change in a measurement environment, and the interference may even cover up an information of the tissue element to be detected, which makes it difficult to extract a weak signal under the interference of a large background noise.

In order to solve the above-mentioned problem, a reference measurement method based on a floating reference theory is proposed. That is, for the tissue element to be detected, there is a source-detection distance at which the absorption and the scattering have a same influence on a diffusely-scattered light intensity and opposite directions, therefore the diffusely-scattered light intensity value emitted from an emission position corresponding to this source-detection distance has zero sensitivity to a change in a concentration of the tissue element to be detected. Such emission position with above characteristics may be referred to as a reference position (or a benchmark position), and the corresponding source-detection distance is a reference distance. Moreover, for the tissue element to be detected, there is also a source-detection distance corresponding to an emission position where the diffusely-scattered light intensity value having a greatest sensitivity to the change in the concentration of the tissue element is emitted. Such emission position with above characteristics may be referred to as a measurement position, and the corresponding source-detection distance is a measurement distance. The diffusely-scattered light intensity value corresponding to the reference distance reflects a response of an interference other than the change in the concentration of the tissue element to be detected in a detection process. The diffusely-scattered light intensity value corresponding to the measurement distance reflects a response of the tissue element to be detected and the response of the interference other than the tissue element to be detected. Therefore, the reference position and/or the measurement position need to be accurately determined.

In a related art, diffusely-scattered light intensity values emitted from a surface of a detected site are generally received by photosensitive surfaces at a limited number of source-detection distances from a center of an incident beam with a central incidence. The limited number of source-detection distances is determined according to an average parameter of most detected objects. On this basis, the reference distance and the measurement distance are further determined from the source-detection distances.

In a process of achieving a concept of the present disclosure, the inventor found that the related art at least has a problem that a detection accuracy is not high.

### SUMMARY

An aspect of the present disclosure provides a method of determining a distance in a non-invasive detection of a tissue element, including: acquiring, for a detected site of a detected object, a first light intensity value for each predetermined wavelength of at least one predetermined wavelength at each source-detection distance of at least two source-detection distances; and determining a first light intensity measurement value and/or a first light intensity reference value from the first light intensity values corresponding to the predetermined wavelength according to an absolute value of a light intensity variation caused by a change in a concentration of a tissue element to be detected, determining a source-detection distance corresponding to the first light intensity measurement value as a measurement distance, and determining a source-detection distance corresponding to the first light intensity reference value as a reference distance, wherein the first light intensity measurement value corresponds to a greatest absolute value of the light intensity variation caused by the change in the concentration of the tissue element to be detected, the first light intensity reference value corresponds to a smallest absolute value of the first light intensity variation caused by the change in the concentration of the tissue element to be detected, and the light intensity variation caused by the change in the concentration of the tissue element to be detected is a variation between the first light intensity value and a corresponding predetermined light intensity value.

Another aspect of the present disclosure provides a method of determining a distance in a non-invasive detection of a tissue element, including: acquiring, for a detected site of a detected object, a tissue optical parameter corresponding to each predetermined wavelength of at least one predetermined wavelength and a tissue optical parameter change relationship caused by a change in a concentration of a tissue element to be detected; and determining each measurement distance and/or each reference distance according to the tissue optical parameter corresponding to each predetermined wavelength and the tissue optical parameter change relationship caused by the change in the concentration of the tissue element to be detected.

Another aspect of the present disclosure provides a non-invasive detection method for a tissue element, including: acquiring, for a detected site of a detected object, a second light intensity measurement value for each predetermined wavelength of at least one predetermined wavelength at a measurement distance, and/or a second light intensity reference value for each predetermined wavelength of at least one predetermined wavelength at a reference distance, wherein each measurement distance and each reference distance are determined according to the method of determining the distance in the non-invasive detection of the tissue element; and determining a concentration of a tissue element to be detected according to the second light intensity measurement value for each predetermined wavelength and/or the second light intensity reference value for each predetermined wavelength.

Another aspect of the present disclosure provides a device of determining a distance in a non-invasive detection of a tissue element, including: a first acquisition module configured to acquire, for a detected site of a detected object, a first light intensity value corresponding to each predetermined wavelength of at least one predetermined wavelength at each source-detection distance of at least two source-detection distances; and a first determination module configured to determine a first light intensity measurement value and/or a first light intensity reference value from the first light intensity values corresponding to the predetermined wavelength according to an absolute value of a light intensity variation caused by a change in a concentration of a tissue element to be detected, determine a source-detection distance corresponding to the first light intensity measurement value as a measurement distance, and determine a source-detection distance corresponding to the first light intensity reference value as a reference distance, wherein the first light intensity measurement value corresponds to a greatest absolute value of the light intensity variation caused by the change in the concentration of the tissue element to be detected, the first light intensity reference value corresponds to a smallest absolute value of the first light intensity variation caused by the change in the concentration of the tissue element to be detected, and the light intensity variation caused by the change in the concentration of the tissue element to be detected is a variation between the first light intensity value and a corresponding predetermined light intensity value.

Another aspect of the present disclosure provides a device of determining a distance in a non-invasive detection of a tissue element, including: a second acquisition module configured to acquire, for a detected site of a detected object, a tissue optical parameter corresponding to each predetermined wavelength of at least one predetermined wavelength and a tissue optical parameter change relationship caused by a change in a concentration of a tissue element to be detected; and a second determination module configured to determine each measurement distance and/or each reference distance according to the tissue optical parameter corresponding to each predetermined wavelength and the tissue optical parameter change relationship caused by the change in the concentration of the tissue element to be detected.

Another aspect of the present disclosure provides a non-invasive detection device for a tissue element, including: a light intensity sensor configured to acquire, for a detected site of a detected object, a second light intensity measurement value for each predetermined wavelength of at least one predetermined wavelength at a measurement distance, and/or a second light intensity reference value for each predetermined wavelength of at least one predetermined wavelength at a reference distance, wherein each measurement distance and each reference distance are determined using the device of determining the distance in the non-invasive detection of the tissue element; and a processor configured to determine a concentration of a tissue element to be detected according to the second light intensity measurement value for each predetermined wavelength and/or the second light intensity reference value for each predetermined wavelength.

Another aspect of the present disclosure provides a wearable apparatus, including a body and the non-invasive detection device for the tissue element described above; the non-invasive detection device for the tissue element is arranged on the body; and the wearable apparatus is worn on the detected site.

Another aspect of the present disclosure provides a non-invasive detection system for a tissue element, including the wearable apparatus described above and a terminal; the processor is communicatively connected with the light intensity sensor and the terminal respectively; the wearable apparatus is worn on the detected site; the light intensity sensor is configured to acquire, for the detected site of the detected object, a second light intensity measurement value for each predetermined wavelength of at least one predetermined wavelength at a measurement distance, and/or a second light intensity reference value for each predetermined wavelength of at least one predetermined wavelength at a reference distance, wherein each measurement distance and each reference distance are determined using the device of determining a distance in a non-invasive detection of a tissue element described above; the processor is configured to process the second light intensity measurement value and/or the second light intensity reference value for each predetermined wavelength to obtain a processed second light intensity measurement value and/or a processed second light intensity reference value for each predetermined wavelength, and transmit the processed second light intensity measurement value and/or the processed second light intensity reference value for each predetermined wavelength to the terminal; and the terminal is configured to determine the concentration of the tissue element to be detected according to the processed second light intensity measurement value and/or the processed second light intensity reference value for each predetermined wavelength.

### BRIEF DESCRIPTION OF THE DRAWINGS

FIG. 1 shows a flowchart of a method of determining a distance in a non-invasive detection of a tissue element according to embodiments of the present disclosure;

FIG. 2 shows a schematic diagram of acquiring a first light intensity value according to embodiments of the present disclosure;

FIG. 3 shows a schematic diagram of acquiring a first light intensity value emitted from a surface of a detected site based on a contact linear photosensitive surface array according to embodiments of the present disclosure;

FIG. 4 shows another schematic diagram of acquiring a first light intensity value emitted from a surface of a detected site based on a contact linear photosensitive surface array according to embodiments of the present disclosure;

FIG. 5 shows a schematic diagram of acquiring a first light intensity value emitted from a surface of a detected site based on a non-contact linear photosensitive surface array according to embodiments of the present disclosure;

FIG. 6 shows another schematic diagram of acquiring a first light intensity value emitted from a surface of a detected site based on a non-contact linear photosensitive surface array according to embodiments of the present disclosure;

FIG. 7 shows a schematic diagram of a linear photosensitive surface array in non-contact with a surface of a detected site according to embodiments of the present disclosure;

FIG. 8 shows another schematic diagram of a linear photosensitive surface array in non-contact with a surface of a detected site according to embodiments of the present disclosure;

FIG. 9 shows a schematic diagram of shielding an interference light according to embodiments of the present disclosure;

FIG. 10 shows another schematic diagram of shielding an interference light according to embodiments of the present disclosure;

FIG. 11 shows still another schematic diagram of shielding an interference light according to embodiments of the present disclosure;

FIG. 12 shows a flowchart of another method of determining a distance in a non-invasive detection of a tissue element according to embodiments of the present disclosure;

FIG. 13 shows a flowchart of still another method of determining a distance in a non-invasive detection of a tissue element according to embodiments of the present disclosure;

FIG. 14 shows a flowchart of a non-invasive detection method for a tissue element according to embodiments of the present disclosure;

FIG. 15 shows a schematic diagram of acquiring a second light intensity value according to embodiments of the present disclosure;

FIG. 16 shows a schematic diagram of acquiring a second light intensity measurement value and a second light intensity reference value emitted from a surface of a detected site based on a contact linear photosensitive surface array according to embodiments of the present disclosure;

FIG. 17 shows another schematic diagram of acquiring a second light intensity measurement value and a second light intensity reference value emitted from a surface of a detected site based on a contact linear photosensitive surface array according to embodiments of the present disclosure;

FIG. 18 shows a schematic diagram of acquiring a second light intensity measurement value and a second light intensity reference value emitted from a surface of a detected site based on a non-contact linear photosensitive surface array according to embodiments of the present disclosure;

FIG. 19 shows another schematic diagram of acquiring a second light intensity measurement value and a second light intensity reference value emitted from a surface of a detected site based on a non-contact linear photosensitive surface array according to embodiments of the present disclosure;

FIG. 20 shows a flowchart of another non-invasive detection method for a tissue element according to embodiments of the present disclosure;

FIG. 21 shows a flowchart of still another non-invasive detection method for a tissue element according to embodiments of the present disclosure;

FIG. 22 shows a schematic structural diagram of a device of determining a distance in a non-invasive detection of a tissue element according to embodiments of the present disclosure;

FIG. 23 shows a schematic structural diagram of a first acquisition module according to embodiments of the present disclosure;

FIG. 24 shows another schematic diagram of a linear photosensitive surface array in non-contact with a surface of a detected site according to embodiments of the present disclosure;

FIG. 25 shows still another schematic diagram of a linear photosensitive surface array in non-contact with a surface of a detected site according to embodiments of the present disclosure;

FIG. 26 shows a schematic structural diagram of a light guide part array according to embodiments of the present disclosure;

FIG. 27 shows a schematic structural diagram of a first flat housing according to embodiments of the present disclosure;

FIG. 28 shows a schematic structural diagram of another light guide part array according to embodiments of the present disclosure;

FIG. 29 shows a schematic structural diagram of another light guide part array according to embodiments of the present disclosure;

FIG. 30 shows a schematic structural diagram of another light guide part array according to embodiments of the present disclosure;

FIG. 31 shows a schematic structural diagram of still another light guide part array according to embodiments of the present disclosure;

FIG. 32 shows another schematic diagram of shielding an interference light according to embodiments of the present disclosure;

FIG. 33 shows another schematic diagram of shielding an interference light according to embodiments of the present disclosure;

FIG. 34 shows still another schematic diagram of shielding an interference light according to embodiments of the present disclosure;

FIG. 35 shows a schematic structural diagram of a non-invasive detection device for a tissue element according to embodiments of the present disclosure;

FIG. 36 shows a schematic structural diagram of a wearable apparatus according to embodiments of the present disclosure; and

FIG. 37 shows a schematic structural diagram of a non-invasive detection system for a tissue element according to embodiments of the present disclosure.

### DETAILED DESCRIPTION OF EMBODIMENTS

Embodiments of the present disclosure will be further described below with reference to the accompanying drawings.

In a process of achieving a concept of the present disclosure, the inventor found that a reference distance and a measurement distance may vary with wavelengths, vary with detected objects, and vary with detected sites. If the reference distance and the measurement distance corresponding to each predetermined wavelength are determined for a detected site of a detected object, it is required to provide photosensitive surfaces at various source-detection distances from a center of an incident beam, which puts forward a high requirement for a production level of a photoelectric detector, or said which depends on the production level of the photoelectric detector. However, limited by a current production level of photoelectric detector, it is difficult to provide photosensitive surfaces at various source-detection distances from the center of the incident beam. The photosensitive surfaces may only be provided at a limited number of source-detection distances according to an average parameter for many detected objects. As a result, it is difficult to accurately determine the reference distance and the measurement distance corresponding to each predetermined wavelength for the detected site of the detected object by using the related art, and thus the detection accuracy is not high.

In order to improve the detection accuracy, the reference distance and/or the measurement distance need to be accurately determined. To solve this problem, the inventor proposes a solution of an arrangement of a photosensitive surface, which will be described below with reference to specific embodiments.

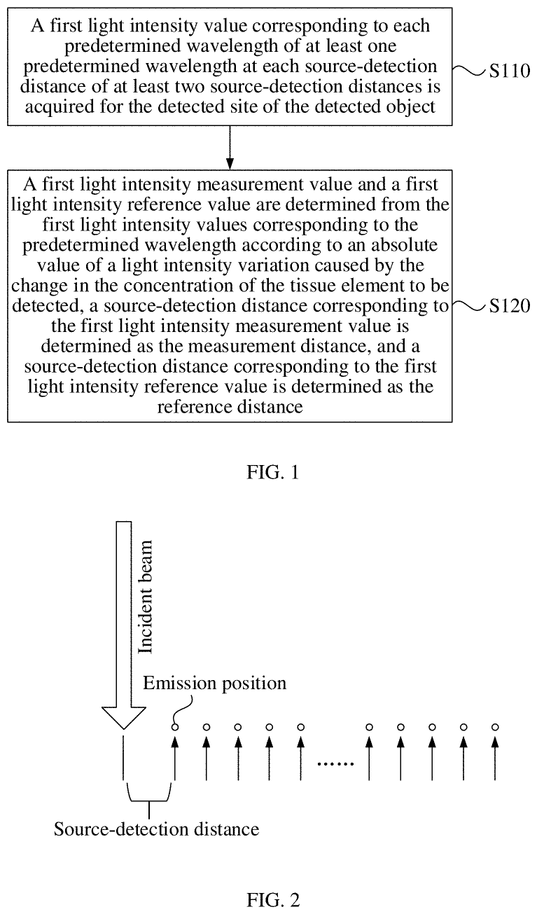

FIG. 1 shows a flowchart of a method of determining a distance in a non-invasive detection of a tissue element according to embodiments of the present disclosure. Such embodiments may be applied to improve a detection accuracy of a concentration of a tissue element to be detected.

As shown in FIG. 1, the method includes operations S110 to S120.

In operation S110, a first light intensity value corresponding to each predetermined wavelength of at least one predetermined wavelength at each source-detection distance of at least two source-detection distances is acquired for the detected site of the detected object.

According to embodiments of the present disclosure, the source-detection distance may represent a distance between a light source and an emission position. The light source here may be understood as a light beam formed on a surface of the detected site, and the emission position may represent a position where the light intensity value is emitted. The light intensity value is emitted from the surface of the detected site after the light beam passes through the detected site. Referring to FIG. 2, FIG. 2 shows a schematic diagram of acquiring a first light intensity value according to embodiments of the present disclosure. The light intensity value described in embodiments of the present disclosure refers to a diffusely-reflected light intensity value, and the light intensity value used to determine the measurement distance and the reference distance in embodiments of the present disclosure is the first light intensity value.

For the detected site of the detected object, at least one first light intensity value corresponding to each predetermined wavelength at each source-detection distance may be acquired. That is, when the detected site of the detected object is determined, for each predetermined wavelength, at least one first light intensity value for the predetermined wavelength at each source-detection distance is acquired. Each first light intensity value here may be a first light intensity value obtained through an in vivo test, a first light intensity value obtained through a Monte Carlo simulation, or a first light intensity value obtained through an in vitro test. Different first light intensity values for the same predetermined wavelength at the same source-detection distance correspond to different concentrations of the tissue element to be detected. That is, at least one first light intensity value for the same predetermined wavelength at the same source-detection distance is acquired, and different first light intensity values correspond to different concentrations of the tissue element to be detected.

If each first light intensity value is the first light intensity value obtained through the in vivo test or the first light intensity value obtained through the in vitro test, then acquiring the first light intensity value corresponding to each predetermined wavelength at each source-detection distance for the detected site of the detected object may be understood as follows. For the detected site of the detected object, the incident beam corresponding to each predetermined wavelength is emitted to the surface of the detected site through a light source entrance. At least one first light intensity value emitted from emission positions arranged at different source-detection distances from the center of each incident beam after the incident beam passes through the detected site is acquired based on a linear photosensitive surface array. It should be noted that if the tissue element to be detected is blood glucose, the above-mentioned in vivo test may include OGTT (Oral Glucose Tolerance Test).

If each first light intensity value is the first light intensity value obtained through the Monte Carlo simulation, then the acquiring the first light intensity value corresponding to each predetermined wavelength at each source-detection distance for the detected site of the detected object may be understood as follows. A tissue optical parameter and a skin structure parameter for each predetermined wavelength in a three-layer skin tissue model are acquired for the detected site of the detected object. Based on the Monte Carlo simulation, the first light intensity value corresponding to each predetermined wavelength at each source-detection distance is determined according to each tissue optical parameter, each skin tissue structure parameter, a tissue optical parameter change relationship caused by a change in the concentration of the tissue element to be detected, at least two predetermined source-detection distances and a predetermined incident photon number. The Monte Carlo simulation may achieve a simulation of an optical propagation path of random scattering in a biological tissue, and a spatial distribution of the diffusely-scattered light intensity value and a distribution of an absorbed photon part in the tissue may be obtained. The three-layer skin tissue model may be understood as including epidermis, dermis and subcutaneous tissue. The tissue optical parameter may include an absorption coefficient, a scattering coefficient, an anisotropy factor and an average refractive index of each skin layer. The skin tissue structure parameter may be understood as a thickness of each layer of skin tissue, that is, a thickness of the epidermis, a thickness of the dermis and a thickness of the subcutaneous tissue. The tissue optical parameter change relationship caused by the change in the concentration of the tissue element to be detected may include an absorption coefficient change relationship caused by the change in the concentration of the tissue element to be detected, and a reduced scattering coefficient change relationship caused by the change in the concentration of the tissue element to be detected. The tissue element to be detected may include blood glucose, fat, and white blood cells, etc.

In operation S120, a first light intensity measurement value and/or a first light intensity reference value are/is determined from the first light intensity values corresponding to the predetermined wavelength according to an absolute value of a light intensity variation caused by the change in the concentration of the tissue element to be detected, a source-detection distance corresponding to the first light intensity measurement value is determined as the measurement distance, and a source-detection distance corresponding to the first light intensity reference value is determined as the reference distance. The first light intensity measurement value is a first light intensity value corresponding to a greatest absolute value of a light intensity variation caused by the change in the concentration of the tissue element to be detected. The first light intensity reference value is a first light intensity value corresponding to a smallest absolute value of a light intensity variation caused by the change in the concentration of the tissue element to be detected. The light intensity variation caused by the change in the concentration of the tissue element to be detected is a variation between the first light intensity value and a corresponding predetermined light intensity value.

According to embodiments of the present disclosure, the measurement distance is the source-detection distance corresponding to the emission position where the diffusely-scattered light intensity value having the greatest sensitivity to the change in the concentration of the tissue element to be detected is emitted, and the reference distance is the source-detection distance corresponding to the emission position where the diffusely-scattered light intensity value having zero sensitivity to the change in the concentration of the tissue element to be detected is emitted, where the sensitivity of the diffusely-scattered light intensity value to the change in the concentration of the tissue element to be detected is a ratio of the light intensity variation to a variation of the concentration of the tissue element to be detected. Therefore, when the variation of the concentration of the tissue element to be detected is determined, the measurement distance is the source-detection distance corresponding to the emission position where the light intensity variation with the greatest absolute value is emitted, and the reference distance is the source-detection distance corresponding to the emission position where the light intensity variation with the smallest absolute value is emitted. The above-mentioned diffusely-reflected light intensity value is the first light intensity value. Based on the above, the first light intensity measurement value and/or the first light intensity reference value may be determined from the first light intensity values corresponding to the predetermined wavelength according to the absolute value of the light intensity variation caused by the change in the concentration of the tissue element to be detected. The source-detection distance corresponding to the first light intensity measurement value is determined as the measurement distance, and the source-detection distance corresponding to the first light intensity reference value is determined as the reference distance. The first light intensity measurement value is the first light intensity value corresponding to the greatest absolute value of the light intensity variation caused by the change in the concentration of the tissue element to be detected. The first light intensity reference value is the first light intensity value corresponding to the smallest absolute value of the light intensity variation caused by the change in the concentration of the tissue element to be detected. The light intensity variation caused by the change in the concentration of the tissue element to be detected is a variation between the first light intensity value and the corresponding predetermined light intensity value. Each predetermined light intensity value may be understood as a light intensity value emitted from the surface of the detected site when the concentration of the tissue element to be detected is a predetermined concentration. If each first light intensity value is the first light intensity value obtained through the in vivo test, then each predetermined light intensity value may be a light intensity value obtained when the detected object is in a fasting state. If each first light intensity value is the first light intensity value obtained by the Monte Carlo simulation or the first light intensity value obtained by the in vitro test, then each predetermined light intensity value may be a light intensity value emitted from the surface of the detected site when the predetermined concentration is zero.

According to embodiments of the present disclosure, the determining the first light intensity measurement value and/or the first light intensity reference value from the first light intensity values corresponding to the predetermined wavelength according to the absolute value of the light intensity variation caused by the change in the concentration of the tissue element to be detected may be understood as follows. For each predetermined wavelength, the first light intensity measurement value may be determined from the first light intensity values corresponding to the predetermined wavelength. Alternatively, the first light intensity measurement value and the first light intensity reference value may be determined from the first light intensity values corresponding to the predetermined wavelength. Alternatively, the first light intensity reference value may be determined from the first light intensity values corresponding to the predetermined wavelength. For at least one predetermined wavelength, there may be the following cases.

In a first case, only the first light intensity measurement value corresponding to each predetermined wavelength is determined. In a second case, the first light intensity measurement value and the first light intensity reference value corresponding to each wavelength are determined. In a third case, at least one first light intensity measurement value corresponding to at least one predetermined wavelength among the predetermined wavelengths is determined, and at least one first light intensity reference value corresponding to at least one other predetermined wavelength among the predetermined wavelengths is determined. In a fourth case, at least one first light intensity measurement value and at least one first light intensity reference value corresponding to at least one predetermined wavelength among the predetermined wavelengths are determined, and at least one first light intensity reference value corresponding to at least one other predetermined wavelength among the predetermined wavelengths is determined. In a fifth case, at least one first light intensity measurement value and at least one first light intensity reference value corresponding to at least one predetermined wavelength among the predetermined wavelengths are determined, and at least one first light intensity measurement value corresponding to at least one other predetermined wavelength among the predetermined wavelengths is determined.

On this basis, from the perspective of the measurement distance and the reference distance, for the at least one predetermined wavelength, there may be the following cases. In a first case, only the measurement distance corresponding to each predetermined wavelength is determined. In a second case, the measurement distance and the reference distance corresponding to each wavelength are determined. In a third case, at least one measurement distance corresponding to at least one predetermined wavelength among the predetermined wavelengths is determined, and at least one reference distance corresponding to at least one other predetermined wavelength among the predetermined wavelengths is determined. In a fourth case, at least one measurement distance and at least one reference distance corresponding to at least one predetermined wavelength among the predetermined wavelengths are determined, and at least one reference distance corresponding to at least one other predetermined wavelength among the predetermined wavelength is determined. In a fifth case, at least one measurement distance and at least one reference distance corresponding to at least one predetermined wavelength among the predetermined wavelengths are determined, and at least one measurement distance corresponding to at least one other predetermined wavelength among the predetermined wavelengths is determined. For each predetermined wavelength, the determination of the measurement distance and/or the reference distance corresponding to the predetermined wavelength may be set according to actual situations, which is not specifically limited herein.

For example, represents the predetermined wavelength, i∈[1, M], M represents a number of the predetermined wavelength, M≥1. ρⱼ represents the source-detection distance, j∈[2, N], N represents a number of the source-detection distances, N≥2. Tₖ represents the concentration of the tissue element to be detected, k∈[1, P], P represents a number of the concentration of the tissue element to be detected, P≥1. A predetermined concentration corresponding to each predetermined light intensity value may be represented by T₀.

When the concentration of the tissue element to be detected is Tₖ, one first light intensity value φ(λᵢ, ρⱼ)∥_{T}_{ₖ} corresponding to each predetermined wavelength λᵢ at each source-detection distance ρⱼ may be acquired for the detected site of the detected object. For each predetermined wavelength λᵢ, N first light intensity values φ(λᵢ, ρⱼ)∥_{λ}_{ᵢ}_{, T}_{ₖ} corresponding to each concentration Tₖ of the tissue element to be detected may be acquired. Accordingly, for each predetermined wavelength λᵢ, P first light intensity value sets may be acquired, and each first light intensity value set includes N first light intensity values φ(λᵢ, ρⱼ)∥_{λ}_{ᵢ}_{, T}_{ₖ}. For each first light intensity value set corresponding to each predetermined wavelength λᵢ, each first light intensity value φ(λᵢ, ρⱼ)∥_{λ}_{ᵢ}_{, T}_{ₖ} in the first light intensity value set may be calculated with the predetermined light intensity value to determine absolute values of N light intensity variations caused by the change in the concentration of the tissue element to be detected. The greatest absolute value of the light intensity variation caused by the change in the concentration of the tissue element to be detected and the smallest absolute value of the light intensity variation caused by the change in the concentration of the tissue element to be detected are determined from the absolute values of the N light intensity variations caused by the change in the concentration of the tissue element to be detected. The change in the concentration of the tissue element to be detected may be represented by Tₖ-T₀. The first light intensity value corresponding to the greatest absolute value of the light intensity variation caused by the change in the concentration of the tissue element to be detected is determined as the first light intensity measurement value, and the first light intensity value corresponding to the smallest absolute value of the light intensity variation caused by the change in the concentration of the tissue element to be detected is determined as the first light intensity reference value. For each predetermined wavelength λᵢ, one first light intensity measurement value and one first light intensity reference value corresponding to each concentration Tₖ of the tissue element to be detected may be acquired. However, for each predetermined wavelength λᵢ, the first light intensity measurement values for different concentrations Tₖ of the tissue element to be detected correspond to the same source-detection distance, and the first light intensity reference values for different concentrations Tₖ of the tissue element to be detected also correspond to the same source-detection distance. The source-detection distance corresponding to the first light intensity measurement value may be determined as the measurement distance, and the source-detection distance corresponding to the first light intensity reference value may be determined as the reference distance.

According to the technical solution of embodiments of the present disclosure, as the first light intensity value corresponding to each predetermined wavelength at each source-detection distance may be acquired for the detected site of the detected object, the first light intensity measurement value and/or the first light intensity reference value may be accurately determined, and then the measurement distance and/or the reference distance may be accurately determined. On this basis, the accurate determination of the measurement distance and/or the reference distance provides a basis for determining the concentration of the tissue element to be detected, thereby improving the detection accuracy.

According to embodiments of the present disclosure, operation 110 may include the following operations. For the detected site of the detected object, the incident beam corresponding to each predetermined wavelength is emitted to the surface of the detected site through a light source entrance. The first light intensity values emitted from emission positions at different source-detection distances from the center of each incident beam after the incident beam passes through the detected site are acquired based on the linear photosensitive surface array. The linear photosensitive surface array includes at least two original photosensitive surfaces, and each original photosensitive surface corresponds to one emission position.

According to embodiments of the present disclosure, in order to accurately determine the measurement distance and/or the reference distance, the linear photosensitive surface array may be used to acquire the first light intensity values emitted from emission positions at different source-detection distances from the center of the incident beam. As the linear photosensitive surface array includes at least two original photosensitive surfaces and each original photosensitive surface corresponds to one emission position, each first light intensity value received by the linear photosensitive surface array is the first light intensity value generated after a corresponding incident beam is incident and passes through a corresponding transmission path. The incident beam and each original photosensitive surface may correspond to one source-detection distance.

According to embodiments of the present disclosure, as the measurement distance and the reference distance may vary with wavelengths, vary with detected objects and vary with detected sites, then for detected site of each detected object, the first light intensity values corresponding to each predetermined wavelength may be acquired by using the above-mentioned manner, so that the reference distance and/or the measurement distance corresponding to each predetermined wavelength may be accurately determined for the detected site of the detected object. In addition, the above-mentioned emission manner of the incident beam and reception manner of the linear photosensitive surface array may greatly reduce the requirements for the photoelectric detector, thereby reducing the manufacturing cost and being easy for implementation.

Based on the above, the implementation is as follows. For the detected site of the detected object, in order to acquire the first light intensity value corresponding to each predetermined wavelength at each source-detection distance, the original photosensitive surfaces may be provided at different source-detection distances from the center of the incident beam. Each original photosensitive surface may receive the first light intensity value emitted from the surface of the detected site at a corresponding source-detection distance, and each original photosensitive surface corresponds to one source-detection distance. The above-mentioned original photosensitive surfaces at different source-detection distances form a linear photosensitive surface array. The linear photosensitive surface array may be a diode array detector, or may be formed by a linear arrangement of different detectors. The linear photosensitive surface array formed by a linear arrangement of different detectors may be understood as follows. The linear photosensitive surface array is formed by a linear arrangement of at least two detectors, each detector is independent, and each detector is provided with a corresponding original photosensitive surface. In addition, the linear photosensitive surface array may be a contact linear photosensitive surface array or a non-contact linear photosensitive surface array. The contact linear photosensitive surface array may be understood as a linear photosensitive surface array in contact with the surface of the detected site, and the non-contact linear photosensitive surface array may be understood as a linear photosensitive surface array in non-contact with the surface of the detected site.

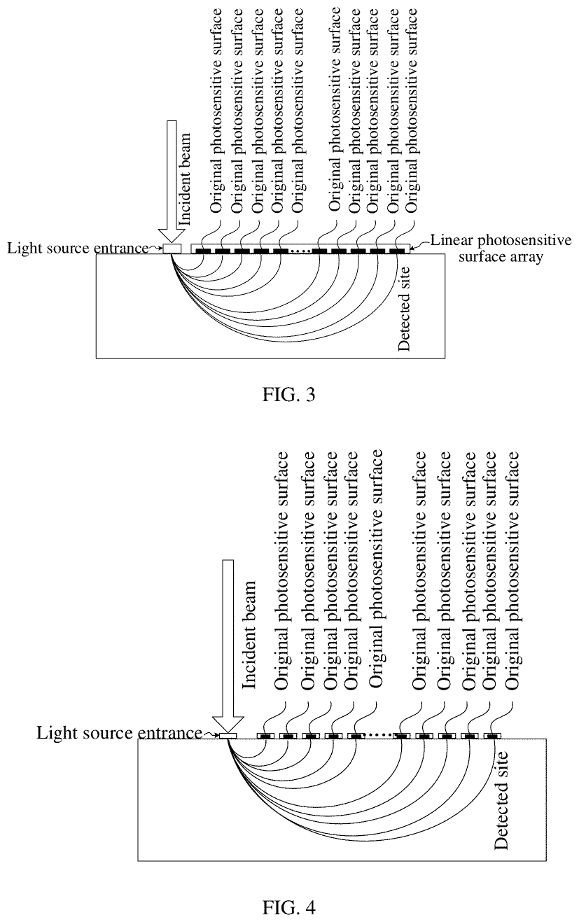

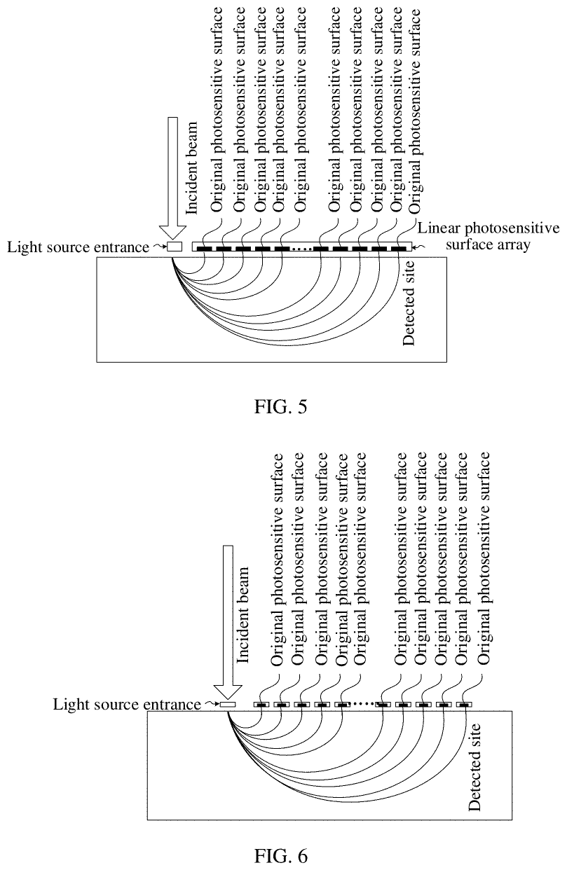

Based on the above, the linear photosensitive surface array may be a contact linear photosensitive surface array, which may be a diode array detector or formed by a linear arrangement of different detectors. The linear photosensitive surface array may be a non-contact linear photosensitive surface array, which may be a diode array detector or formed by a linear arrangement of different detectors. For example, as shown in FIG. 3, FIG. 3 shows a schematic diagram of acquiring a first light intensity value emitted from a surface of a detected site based on a contact linear photosensitive surface array according to embodiments of the present disclosure. The contact linear photosensitive surface array is a diode array detector. As shown in FIG. 4, FIG. 4 shows another schematic diagram of acquiring a first light intensity value emitted from a surface of a detected site based on a contact linear photosensitive surface array according to embodiments of the present disclosure. The contact linear photosensitive surface array is formed by a linear arrangement of different detectors. As shown in FIG. 5, FIG. 5 shows a schematic diagram of acquiring a first light intensity value emitted from a surface of a detected site based on a non-contact linear photosensitive surface array according to embodiments of the present disclosure. The non-contact linear photosensitive surface array is a diode array detector. As shown in FIG. 6, FIG. 6 shows another schematic diagram of acquiring a first light intensity value emitted from a surface of a detected site based on a non-contact linear photosensitive surface array according to embodiments of the present disclosure. The non-contact linear photosensitive surface array is formed by a linear arrangement of different detectors.

According to embodiments of the present disclosure, as the first light intensity value corresponding to each predetermined wavelength at each source-detection distance may be acquired based on the linear photosensitive surface, the first light intensity measurement value and/or the first light intensity reference value may be accurately determined, and then the measurement distance and the reference distance may be accurately determined. The above-mentioned emission manner of the incident beam and reception manner of the linear photosensitive surface array may greatly reduce the requirements for the photoelectric detector, thereby reducing the manufacturing cost and being easy for implementation.

As shown in FIG. 3 to FIG. 6, according to embodiments of the present disclosure, the linear photosensitive surface array is a diode array detector or formed by a linear arrangement of different detectors.

According to embodiments of the present disclosure, as shown in FIG. 3 and FIG. 5, the linear photosensitive surface array may be a diode array detector. As shown in FIG. 4 and FIG. 6, the linear photosensitive surface array may be formed by a linear arrangement of different detectors, and each detector is provided with a corresponding original photosensitive surface.

As shown in FIG. 3 to FIG. 8, according to embodiments of the present disclosure, the light source entrance is in contact or non-contact with the surface of the detected site; and/or the linear photosensitive surface array is in contact or non-contact with the surface of the detected site.

According to embodiments of the present disclosure, a form of the non-invasive detection of the tissue element may include a contact detection and a non-contact detection. The contact detection may be implemented to prevent interference light from being received by the linear photosensitive surface array, thereby further improving the detection accuracy. The non-contact detection may be implemented to avoid an influence of interfering factors such as temperature and pressure on the change of the light intensity value, thereby further improving the detection accuracy.

If the light source entrance is arranged in contact with the surface of the detected site and/or the linear photosensitive surface array is arranged in contact with the surface of the detected site, it may be considered that the non-invasive detection of the tissue element is the contact detection. The above may prevent the interference light from being received by the linear photosensitive surface array, thereby further improving the detection accuracy.

If the light source entrance is arranged in non-contact with the surface of the detected site, and the linear photosensitive surface array is arranged in non-contact with the surface of the detected site, then the form of the non-invasive detection of tissue element may be determined according to whether the incident beam from the light source entrance is transmitted through a light guide part array and whether the linear photosensitive surface array receives the first light intensity values through the light guide part array, and whether the light guide part array is in contact with the surface of the detected site if the incident beam is transmitted through the light guide part array and the first light intensity values are received through the light guide part array. The light guide part array includes a first end of the light guide part array and a second end of the light guide part array. A distance between the first end of the light guide part array and the surface of the detected site is greater than a distance between the second end of the light guide part array and the surface of the detected site. The first end of the light guide part array and the second end of the light guide part array are opposite end faces. The second end of the light guide part array is in contact or non-contact with the surface of the detected site. After the incident beam is transmitted through the light source entrance to the first end of the light guide part array and then transmitted to the detected site through the second end of the light guide part array, a beam emitted from the surface of the detected site may enter the light guide part array through the second end of the light guide part array and then is transmitted to the first end of the light guide part array

If the light source entrance is arranged in non-contact with the surface of the detected site, and the linear photosensitive surface array is arranged in non-contact with the surface of the detected site, and the incident beam from the light source entrance is not transmitted through the light guide part array and the linear photosensitive surface array receives the first light intensity values that do not pass through the light guide part array, the form of the non-invasive detection of tissue element may be considered as the non-contact detection. If the incident beam from the light source entrance is transmitted through the light guide part array and the linear photosensitive surface array receives the first light intensity values through the light guide part array, the light source entrance needs to be arranged in contact with the first end of the light guide part array and the linear photosensitive surface array needs to be arranged at the first end of the light guide part array in order to achieve the non-contact between the light source entrance and the surface of the detected site and the non-contact between the linear photosensitive surface array and the surface of the detected site. On this basis, the form of the non-invasive detection of tissue element is determined according to whether the second end of the light guide part array is in contact with the surface of the detected site. That is, if the second end of the light guide part array is in contact with the surface of the detected site, it may be considered that the form of the non-invasive detection of tissue element is the contact detection. If the second end of the light guide part array is in non-contact with the surface of the detected site, it may be considered that the form of the non-invasive detection of tissue element is the non-contact detection.

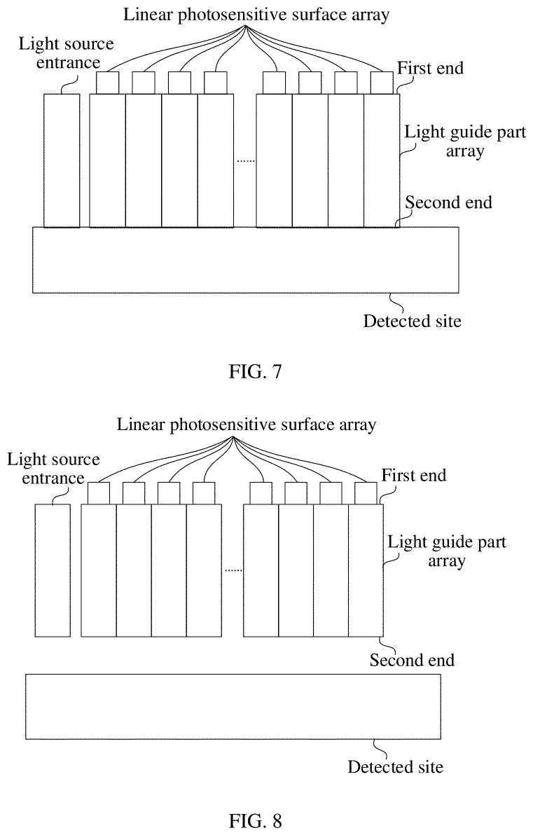

To sum up, the contact detection may include the following two manners. In a first manner, the light source entrance is in contact with the surface of the detected site, and/or the linear photosensitive surface array is in contact with the surface of the detected site, as shown in FIG. 3 and FIG. 4. In a second manner, the light source entrance is in contact with the first end of the light guide part array, and the linear photosensitive surface array is arranged at the first end of the light guide part array, and the second end of the light guide part array is in contact with the surface of the detected site, as shown in FIG. 7. FIG. 7 shows a schematic diagram of the linear photosensitive surface array in non-contact with the surface of the detected site according to embodiments of the present disclosure. In FIG. 7, the second end of the light guide part array is in contact with the surface of the detected site.

The non-contact detection may include the following two manners. In a first manner, the light source entrance and the linear photosensitive surface array are in non-contact with the surface of the detected site, the incident beam from the light source entrance is not transmitted through the light guide part array, and the linear photosensitive surface array receives the first light intensity values that do not pass through the light guide part array, as shown in FIG. 5 and FIG. 6. In FIG. 5 and FIG. 6, the incident beam from the light source entrance is not transmitted through the light guide part array, and the linear photosensitive surface array receives the first light intensity values that do not pass through the light guide part array. In a second manner, the light source entrance is in contact with the first end of the light guide part array, and the linear photosensitive surface array is arranged at the first end of the light guide part array, and the second end of the light guide part array is in non-contact with the surface of the detected site, as shown in FIG. 8. FIG. 8 shows another schematic diagram of the linear photosensitive surface array in non-contact with the surface of the detected site according to embodiments of the present disclosure. In FIG. 8, the second end of the light guide part array is in non-contact with the surface of the detected site.

According to embodiments of the present disclosure, the light source entrance and the linear photosensitive surface array may be integral or separate.

As shown in FIG. 7 and FIG. 8, according to embodiments of the present disclosure, the non-contact between the light source entrance and the surface of the detected site and the non-contact between the linear photosensitive surface array and the surface of the detected site may be achieved by an arrangement that the light source entrance is in contact with the first end of the light guide part array, the linear photosensitive surface array is arranged at the first end of the light guide part array, the second end of the light guide part array is in contact or non-contact with the surface of the detected site, and the first end of the light guide part array and the second end of the light guide part array are opposite end faces.

According to embodiments of the present disclosure, in order to achieve the non-contact between the light source entrance and the surface of the detected site, and the non-contact between the linear photosensitive surface array and the surface of the detected site, the light source entrance may be arranged in contact with the first end of the light guide part array, and the linear photosensitive surface array may be arranged at the first end of the light guide part array. The first end of the light guide part array in non-contact with the surface of the detected site may be provided with the linear photosensitive surface array and may be in contact with the light source entrance. The second end of the light guide part array opposite to the first end of the light guide part array may be in contact with the surface of the detected site, or may be in non-contact with the surface of the detected site, which may be set according to actual situations and is not specifically limited here.

If the light source entrance is in contact with the first end of the light guide part array, the linear photosensitive surface array is arranged at the first end of the light guide part array, and the second end of the light guide part array is in contact with the surface of the detected site, it may be considered that the form of the non-invasive detection of tissue element is the contact detection, as shown in FIG. 7. If the light source entrance is in contact with the first end of the light guide part array, the linear photosensitive surface array is arranged at the first end of the light guide part array, and the second end of the light guide part array is in non-contact with the surface of the detected site, it may be considered that the form of the non-invasive detection of tissue element is the non-contact detection, as shown in FIG. 8.

According to embodiments of the present disclosure, the light guide part array includes at least one emission light guide part and a receiving light guide part array. The receiving light guide part array includes at least two receiving light guide parts. A distance between first ends of two adjacent receiving light guide parts is greater than or equal to a distance between second ends of two adjacent receiving light guide parts. A cross-sectional area of the first end of each receiving light guide part is greater than or equal to a cross-sectional area of the second end of each receiving light guide part.

According to embodiments of the present disclosure, in order to improve the detection accuracy, it is necessary to improve a spatial resolution and a light intensity signal-to-noise ratio. The spatial resolution may be improved by setting numerous and dense source-detection distances, and the light intensity signal-to-noise ratio may be improved by selecting a photoelectric detector (i.e., the original photosensitive surface) with a large photosensitive area. In order to meet the requirements of above-mentioned both aspects, the light guide part array may be arranged as a fan-shaped light guide part array, that is, the distance between the first ends of two adjacent receiving light guide parts is greater than the distance between the second ends of two adjacent receiving light guide parts. In this way, the first end of the light guide part array may be provided with a photoelectric detector with a large photosensitive area, and the second end of the light guide part array may be provided with numerous and dense source-detection distances.

According to embodiments of the present disclosure, if a photoelectric detector with a large size is selected to further improve the light intensity signal-to-noise ratio, the end faces of the receiving light guide part may be set to be gradient, that is, each receiving light guide part may be set so that the cross-sectional area of the first end of the receiving light guide part is greater than the cross-sectional area of the second end of the receiving light guide part.

According to embodiments of the present disclosure, the light source entrance and the linear photosensitive surface array are in non-contact with the surface of the detected site. Before acquiring, based on the linear photosensitive surface array, the first light intensity values that are emitted from the emission positions at different source-detection distances from the center of each incident beam after the incident beam passes through the detected site, the method may further include an operation of shielding interference light.

According to embodiments of the present disclosure, after the incident beam is transmitted to the detected site, a part of the incident beam may be directly reflected on the surface of the detected site to form a surface-reflected light, and a part of the incident beam passes through the detected site and a diffusely-scattered light (i.e., the first light intensity value) is emitted from the surface of the detected site. The surface-reflected light does not interact with the tissue and therefore may not carry valid information. The valid information may be understood as a response caused by the change in the concentration of the tissue element to be detected in the detection process. Therefore, the surface-reflected light may be regarded as the interference light. The diffusely-scattered light interacts with the skin tissue and carries the valid information, and thus may be regarded as valid light.

If the light source entrance is in non-contact with the surface of the detected site, the surface-reflected light may be generated. Based on this, in order to further improve the detection accuracy, the interference light may be shielded before acquiring, based on the linear photosensitive surface array, the first light intensity values that are emitted from the emission positions arranged at different source-detection distances from the center of each incident beam after the incident beam passes through the detected site, so that the first light intensity values emitted from the surface of the detected site after each incident beam passes through the detected site are acquired based on the linear photosensitive surface array. The interference light may be shielded in the following two manners.

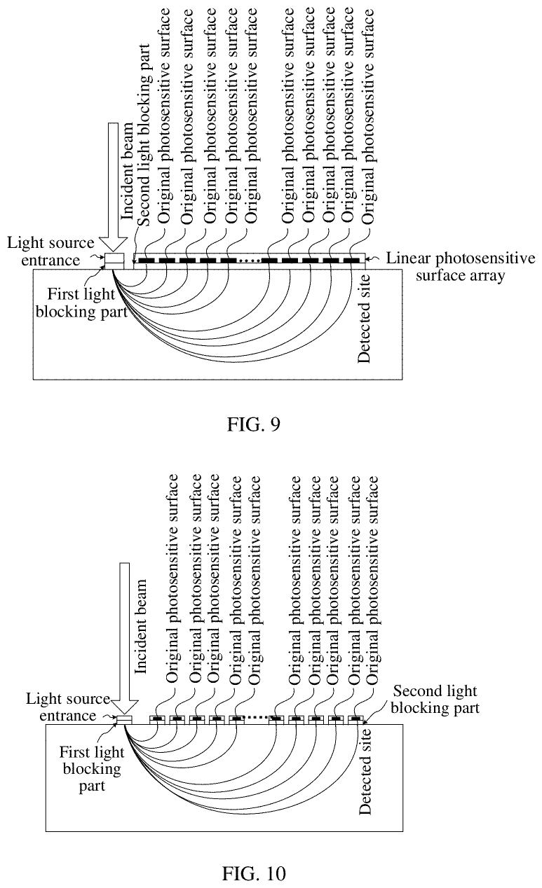

In a first manner, if the light source entrance and the linear photosensitive surface array are in non-contact with the surface of the detected site, and the incident beam from the light source entrance is not transmitted through the light guide part array and the linear photosensitive surface array receives the first light intensity values that do not pass through the light guide part array, a first light blocking part may be provided in a gap region between the light source entrance and the surface of the detected site, and/or a second light blocking part may be provided in a gap region between the linear photosensitive surface array and the surface of the detected site. The first light blocking part is in contact with the surface of the detected site. The light source entrance is arranged inside the first light blocking part. The first light blocking part is integral with the light source entrance, or the first light blocking part is separate from the light source entrance. The second light blocking part is in contact with the surface of the detected site. The linear photosensitive surface array is arranged inside the second light blocking part. The second light blocking part is integral with the linear photosensitive surface array, or the second light blocking part is separate from the linear photosensitive surface array. Both or either of the first light blocking part and the second light blocking part may be provided. As shown in FIG. 9, FIG. 9 shows a schematic diagram of shielding the interference light according to embodiments of the present disclosure. As shown in FIG. 10, FIG. 10 shows another schematic diagram of shielding the interference light according to embodiments of the present disclosure.

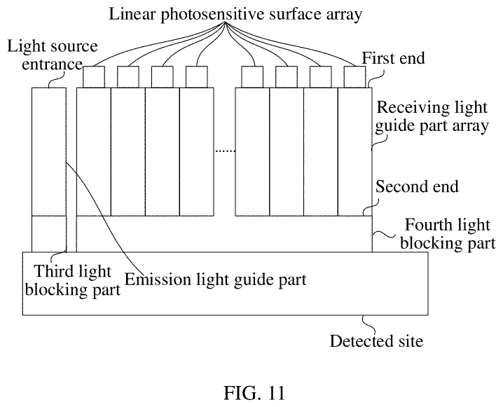

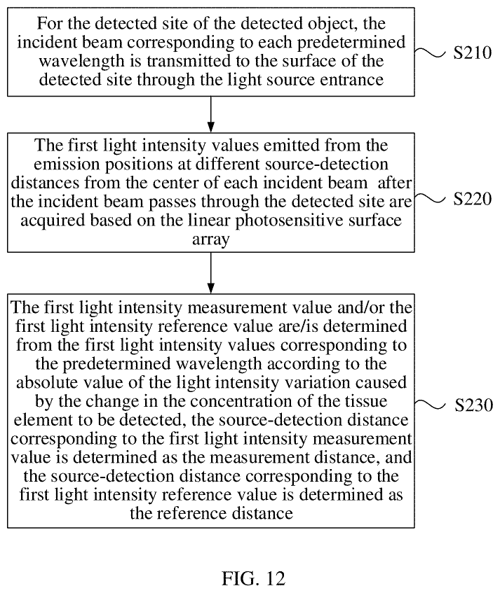

In a second manner, if the light source entrance is in contact with the first end of the light guide part array, the linear photosensitive surface array is provided at the first end of the light guide part array, and the second end of the light guide part array is in non-contact with the surface of the detected site, then a third light blocking part may be provided in a gap region between the emission light guide part and the surface of the detected site, and/or a fourth light blocking part may be provided in a gap region between the receiving light guide part array and the surface of the detected site. A first end of the third light blocking part is in contact with the second end of the emission light guide part, a second end of the third light blocking part is in contact with the surface of the detected site, and the second end of the third light blocking part and the first end of the third light blocking part are opposite end faces. A distance between the first end of the third light blocking part and the surface of the detected site is greater than a distance between the second end of the third light blocking part and the surface of the detected site. A first end of the fourth light blocking part is in contact with the second end of the receiving light guide part array, a second end of the fourth light blocking part is in contact with the surface of the detected site, and the second end of the fourth light blocking part and the first end of the fourth light blocking part are opposite end faces. A distance between the first end of the fourth light blocking part and the surface of the detected site is greater than a distance between the second end of the fourth light blocking part and the surface of the detected site. The light guide part array includes the emission light guide part and the receiving light guide part array. Both or either of the third light blocking part and the fourth light blocking part may be provided. As shown in FIG. 11, FIG. 11 shows a schematic diagram of still another schematic diagram of shielding the interference light according to embodiments of the present disclosure.

According to embodiments of the present disclosure, the interference light is shielded before acquiring the first light intensity values emitted from the surface of the detected site after each incident beam passes through the detected site, so that only the diffusely-scattered light is received by the linear photosensitive surface array. As the diffusely-scattered light carries the valid information, the detection accuracy may be further improved.

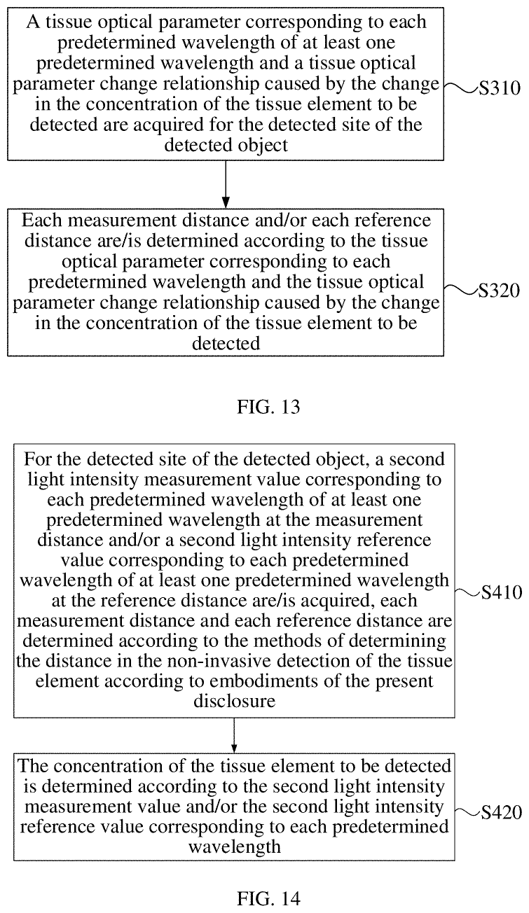

FIG. 12 shows a flowchart of another method of determining a distance in a non-invasive detection of a tissue element according to embodiments of the present disclosure. Such embodiments may be applied to improve the detection accuracy of the concentration of the tissue element to be detected.

As shown in FIG. 12, the method includes operations S210 to S230.

In operation S210, for the detected site of the detected object, the incident beam corresponding to each predetermined wavelength is transmitted to the surface of the detected site through the light source entrance.

Embodiments of the present disclosure provide at least two source-detection distances and at least one predetermined wavelength.

In operation S220, the first light intensity values emitted from the emission positions at different source-detection distances from the center of each incident beam after the incident beam passes through the detected site are acquired based on the linear photosensitive surface array.

According to embodiments of the present disclosure, the linear photosensitive surface array includes at least two original photosensitive surfaces, and each original photosensitive surface corresponds to one emission position. The linear photosensitive surface array may be a diode array detector or formed by a linear arrangement of different detectors. The light source entrance is in contact or non-contact with the surface of the detected site, and/or the linear photosensitive surface array is in contact or non-contact with the surface of the detected site. The non-contact between the light source entrance and the surface of the detected site and the non-contact between the linear photosensitive surface array and the surface of the detected site may be achieved by an arrangement that the light source entrance is in contact with the first end of the light guide part array, the linear photosensitive surface array is arranged at the first end of the light guide part array, the second end of the light guide part array is in contact or non-contact with the surface of the detected site, and the second end of the light guide part array and the first end of the light guide part array are opposite end faces. If the light source entrance and the linear photosensitive surface array are in non-contact with the surface of the detected site, the method may further include an operation of shielding interference light before operation S220.

In operation S230, the first light intensity measurement value and/or the first light intensity reference value are/is determined from the first light intensity values corresponding to the predetermined wavelength according to the absolute value of the light intensity variation caused by the change in the concentration of the tissue element to be detected, the source-detection distance corresponding to the first light intensity measurement value is determined as the measurement distance, and the source-detection distance corresponding to the first light intensity reference value is determined as the reference distance.

According to embodiments of the present disclosure, the first light intensity measurement value is the first light intensity value corresponding to the greatest absolute value of the light intensity variation caused by the change in the concentration of the tissue element to be detected, and the first light intensity reference value is the first light intensity value corresponding to the smallest absolute value of the light intensity variation caused by the change in the concentration of the tissue element to be detected. The light intensity variation caused by the change in the concentration of the tissue element to be detected is the variation between the first light intensity value and a corresponding predetermined light intensity value.

According to the technical solution of embodiments of the present disclosure, as the first light intensity value corresponding to each predetermined wavelength at each source-detection distance may be acquired by the linear photosensitive surface array, the first light intensity measurement value and the first light intensity reference value may be accurately determined, and then the measurement distance and the reference distance may be accurately determined. On this basis, the accurate determination of the measurement distance and the reference distance provides a basis for determining the concentration of the tissue element to be detected, thereby improving the detection accuracy. In addition, the emission manner of the incident beam and reception manner of the linear photosensitive surface array may greatly reduce the requirements for the photoelectric detector, thereby reducing the manufacturing cost and being easy for implementation.

FIG. 13 shows a flowchart of still another method of determining a distance in a non-invasive detection of a tissue element according to embodiments of the present disclosure. Such embodiments may be applied to improve the detection accuracy of the concentration of the tissue element to be detected.

As shown in FIG. 13, the method includes operations S310 to S320.

In operation S310, a tissue optical parameter corresponding to each predetermined wavelength of at least one predetermined wavelength and a tissue optical parameter change relationship caused by the change in the concentration of the tissue element to be detected are acquired for the detected site of the detected object.

In operation S320, each measurement distance and/or each reference distance are/is determined according to the tissue optical parameter corresponding to each predetermined wavelength and the tissue optical parameter change relationship caused by the change in the concentration of the tissue element to be detected.

According to embodiments of the present disclosure, for a body, a body tissue may be simplified into a complex medium constituted by a scattering body and a scattering background, when an incident beam enters the tissue, absorption and scattering may occur, the absorption may directly cause an attenuation of light energy, and the scattering may affect a light energy distribution by changing a transmitting direction of photon, a light intensity value of diffusely-scattered light emitted from the surface of the detected site is a result of a combined effect of the absorption and the scattering. The absorption and the scattering may be reflected by the tissue optical parameter. According to the above, the measurement distance and the reference distance are determined by the absorption and the scattering in different cases. Therefore, for the detected site of the detected object, in order to acquire the measurement distance and/or the reference distance corresponding to each predetermined wavelength, the tissue optical parameter corresponding to each predetermined wavelength and the tissue optical parameter change relationship caused by the change in the concentration of the tissue element to be detected may be acquired. For the tissue optical parameter and the tissue optical parameter change relationship caused by the change in the concentration of the tissue element to be detected, reference may be made to the above description.

After the tissue optical parameter corresponding to each predetermined wavelength is acquired, the measurement distance and/or the reference distance corresponding to each predetermined wavelength may be determined according to the tissue optical parameter corresponding to each predetermined wavelength and the tissue optical parameter change relationship caused by the change in the concentration of the tissue element to be detected. That is, for each predetermined wavelength, the measurement distance and the reference distance corresponding to the predetermined wavelength may be determined according to the tissue optical parameter corresponding to the predetermined wavelength. The measurement distance and/or the reference distance corresponding to each predetermined wavelength may be determined according to the tissue optical parameter corresponding to each predetermined wavelength and the tissue optical parameter change relationship caused by the change in the concentration of the tissue element to be detected based on the floating reference theory. The premise is that the detected site of the detected object is determined. In other words, the above-mentioned measurement distance and the reference distance corresponding to each predetermined wavelength correspond to the detected site of the detected object.

FIG. 14 shows a flowchart of a non-invasive detection method for a tissue element according to embodiments of the present disclosure. Such embodiments may be applied to improve the detection accuracy of the concentration of the tissue element to be detected.

As shown in FIG. 14, the method includes operations S410 to S420.

In operation S410, for the detected site of the detected object, a second light intensity measurement value corresponding to each predetermined wavelength of at least one predetermined wavelength at the measurement distance and/or a second light intensity reference value corresponding to each predetermined wavelength of at least one predetermined wavelength at the reference distance are/is acquired. Each measurement distance and each reference distance are determined according to the methods of determining the distance in the non-invasive detection of the tissue element according to embodiments of the present disclosure.

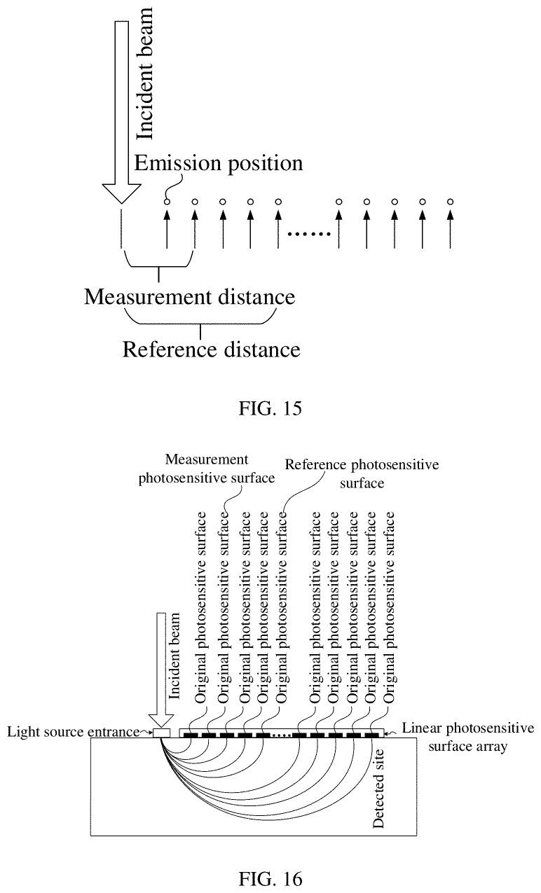

According to embodiments of the present disclosure, in order to determine the concentration of the tissue element to be detected, the second light intensity measurement value and/or the second light intensity reference value corresponding to each predetermined wavelength may be acquired for the detected site of the detected object. The second light intensity measurement value may be a second light intensity value corresponding to each predetermined wavelength at the measurement distance. The second light intensity reference value may be a second light intensity value corresponding to each predetermined wavelength at the reference distance. As shown in FIG. 15, FIG. 15 shows a schematic diagram of acquiring the second light intensity value according to embodiments of the present disclosure. Different predetermined wavelengths may correspond to the same measurement distance or different measurement distances, and different predetermined wavelengths may correspond to the same reference distance or different reference distances. Each measurement distance and each reference distance may be determined according to the methods described in embodiments of the present disclosure, including the following two manners.

In a first manner, for the detected site of the detected object, each measurement distance and each reference distance may be determined by analyzing, for each predetermined wavelength, the acquired at least one first light intensity value corresponding to each source-detection distance. That is, for each predetermined wavelength, at least one first light intensity value corresponding to each source-detection distance is acquired, and each first light intensity value is analyzed, so as to determine a measurement distance and/or a reference distance corresponding to the predetermined wavelength. In other words, for the detected site of the detected object, at least one first light intensity value corresponding to each predetermined wavelength at each source-detection distance is acquired. The first light intensity measurement value and/or the first light intensity reference value may be determined from the first light intensity values corresponding to the predetermined wavelength according to the absolute value of the light intensity variation caused by the change in the concentration of the tissue element to be detected, the source-detection distance corresponding to the first light intensity measurement value is determined as the measurement distance, and the source-detection distance corresponding to the first light intensity reference value is determined as the reference distance. The acquiring at least one first light intensity value corresponding to each predetermined wavelength at each source-detection distance for the detected site of the detected object may be understood as follows. For the detected site of the detected object, the incident beam corresponding to each predetermined wavelength is transmitted to the surface of the detected site through the light source entrance. The first light intensity values emitted from the emission positions at different source-detection distances from the center of each incident beam after the incident beam passes through the detected site are acquired based on the linear photosensitive surface array. The linear photosensitive surface array includes at least two original photosensitive surfaces, and each original photosensitive surface corresponds to one emission position.

In a second manner, for the detected site of the detected object, the tissue optical parameter corresponding to each predetermined wavelength and the tissue optical parameter change relationship caused by the change in the concentration of the tissue element to be detected are acquired. Each measurement distance and/or each reference distance may be determined according to the tissue optical parameter for each predetermined wavelength and the tissue optical parameter change relationship caused by the change in the concentration of the tissue element to be detected.

According to embodiments of the present disclosure, the acquiring the second light intensity measurement value corresponding to each predetermined wavelength at the measurement distance and/or the second light intensity reference value corresponding to each predetermined wavelength at the reference distance for the detected site of the detected object may be understood as follows. For each predetermined wavelength, the second light intensity measurement value corresponding to the predetermined wavelength at the measurement distance may be acquired; or the second light intensity reference value corresponding to the predetermined wavelength at the reference distance may be acquired; or the second light intensity measurement value corresponding to the predetermined wavelength at the measurement distance and the second light intensity reference value corresponding to the predetermined wavelength at the reference distance may be acquired.

For at least one predetermined wavelength, there may be the following cases. In a first case, only the second light intensity measurement value corresponding to each predetermined wavelength is acquired. In a second case, the second light intensity measurement value and the second light intensity reference value corresponding to each wavelength are acquired. In a third case, at least one second light intensity measurement value corresponding to at least one predetermined wavelength among the predetermined wavelengths is acquired, and at least one second light intensity reference value corresponding to at least one other predetermined wavelength among the predetermined wavelengths is acquired. In a fourth case, at least one second light intensity measurement value and at least one second light intensity reference value corresponding to at least one predetermined wavelength among the predetermined wavelengths are acquired, and at least one second light intensity reference value corresponding to at least one other predetermined wavelength among the predetermined wavelengths is acquired. In a fifth case, at least one second light intensity measurement value and at least one second light intensity reference value corresponding to at least one predetermined wavelength among the predetermined wavelengths are acquired, and at least one second light intensity measurement value corresponding to at least one other predetermined wavelength among the predetermined wavelengths is acquired. For each predetermined wavelength, the acquiring the second light intensity measurement value and/or the second light intensity reference value corresponding to the predetermined wavelength may be set according to actual situations, which is not specifically limited herein.

According to embodiments of the present disclosure, the second light intensity measurement value and/or the second light intensity reference value may be accurately determined, and the concentration of the tissue element to be detected may be determined according to the accurately determined second light intensity measurement value and/or second light intensity reference value, so that the detection accuracy may be improved.

In operation S420, the concentration of the tissue element to be detected is determined according to the second light intensity measurement value and/or the second light intensity reference value corresponding to each predetermined wavelength.

According to embodiments of the present disclosure, after each light intensity value corresponding to each predetermined wavelength is acquired, the concentration of the tissue element to be detected may be determined according to the second light intensity measurement value and/or the second light intensity reference value corresponding to each predetermined wavelength. That is, for at least one predetermined wavelength, there may be the following cases.

In a first case, only the second light intensity measurement value corresponding to each predetermined wavelength is acquired. In this case, the concentration of the tissue element to be detected may be determined according to the second light intensity measurement value corresponding to each predetermined wavelength.

In a second case, the second light intensity measurement value and the second light intensity reference value corresponding to each wavelength are acquired. In this case, the concentration of the tissue element to be detected may be determined using a difference operation. That is, for each predetermined wavelength, a difference operation is performed between the second light intensity measurement value and the second light intensity reference value corresponding to the predetermined wavelength, so as to obtain a light intensity difference value. The concentration of the tissue element to be detected is determined according to the light intensity difference value corresponding to each predetermined wavelength. The difference operation is performed because the second light intensity reference value corresponding to the reference distance reflects the response caused by the interference other than the change in the concentration of the tissue element to be detected in the detection process, while the second light intensity measurement value corresponding to the measurement distance reflects the response of the tissue element to be detected and the response of the interference other than the tissue element to be detected. Therefore, a reference-measurement may be used, that is, the second light intensity reference value corresponding to the reference distance may be used to correct the second light intensity measurement value corresponding to the measurement distance, so as to eliminate common mode interference to the greatest extent, thereby further improving the detection accuracy.

In a third case, at least one second light intensity measurement value corresponding to at least one predetermined wavelength among the predetermined wavelengths is acquired, and at least one second light intensity reference value corresponding to at least one other predetermined wavelength among the predetermined wavelengths is acquired. In this case, the concentration of the tissue element to be detected may be determined according to the at least one second light intensity measurement value and the at least one second light intensity reference value corresponding to respective predetermined wavelength(s).

In a fourth case, at least one second light intensity measurement value and at least one second light intensity reference value corresponding to at least one predetermined wavelength among the predetermined wavelengths are acquired, and at least one second light intensity reference value corresponding to at least one other predetermined wavelength among the predetermined wavelengths is acquired. In this case, the concentration of the tissue element to be detected may be determined using a difference operation. That is, for a predetermined wavelength corresponding to which the second light intensity measurement value and the second light intensity reference value are acquired, a difference operation is performed between the second light intensity measurement value and the second light intensity reference value corresponding to the predetermined wavelength, so as to obtain a light intensity difference value. The concentration of the tissue element to be detected is determined according to the light intensity difference value(s) corresponding to the at least one predetermined wavelength among the predetermined wavelengths and the at least one second light intensity reference value corresponding to the at least one other predetermined wavelength among the predetermined wavelengths. Therefore, the reference-measurement may be used, that is, the second light intensity reference value corresponding to the reference distance may be used to correct the second light intensity measurement value corresponding to the measurement distance, so as to eliminate common mode interference to the greatest extent, thereby further improving the detection accuracy.

In a fifth case, at least one second light intensity measurement value and at least one second light intensity reference value corresponding to at least one predetermined wavelength among the predetermined wavelengths are acquired, and at least one second light intensity measurement value corresponding to at least one other predetermined wavelength among the predetermined wavelengths is acquired. In this case, the concentration of the tissue element to be detected may be determined using a difference operation. That is, for a predetermined wavelength corresponding to which the second light intensity measurement value and the second light intensity reference value are acquired, a difference operation is performed between the second light intensity measurement value and the second light intensity reference value corresponding to the predetermined wavelength, so as to obtain a light intensity difference value. The concentration of the tissue element to be detected is determined according to the light intensity difference value(s) corresponding to the at least one predetermined wavelength among the predetermined wavelengths and the at least one second light intensity measurement value corresponding to the at least one other predetermined wavelength among the predetermined wavelengths. Therefore, the reference-measurement may be used, that is, the second light intensity reference value corresponding to the reference distance may be used to correct the second light intensity measurement value corresponding to the measurement distance, so as to eliminate common mode interference to the greatest extent, thereby further improving the detection accuracy.

According to the technical solution of embodiments of the present disclosure, the measurement distance and/or the reference distance corresponding to each predetermined wavelength may be accurately acquired for the detected site of the detected object. Therefore, the second light intensity measurement value and/or the second light intensity reference value may be accurately determined according to the accurately determined measurement distance and/or reference distance. As the concentration of the tissue element to be detected is determined according to the accurately determined second light intensity measurement value and/or the second light intensity reference value, the detection accuracy may be improved.

According to embodiments of the present disclosure, operation 410 may include the following operations. For the detected site of the detected object, the incident beam corresponding to each predetermined wavelength is transmitted to the surface of the detected site through the light source entrance. The second light intensity measurement value emitted from the surface of the detected site after each incident beam passes through the detected site is acquired based on a measurement photosensitive surface corresponding to each predetermined wavelength, wherein the source-detection distance between each measurement photosensitive surface and the center of the incident beam is a corresponding measurement distance; and/or the second light intensity reference value emitted from the surface of the detected site after each incident beam passes through the detected site is acquired based on a reference photosensitive surface corresponding to each predetermined wavelength, wherein the source-detection distance between each reference photosensitive surface and the center of the incident beam is a corresponding reference distance.

According to embodiments of the present disclosure, the source-detection distance between each measurement photosensitive surface and the center of the incident beam is the corresponding measurement distance, and the source-detection distance between each reference photosensitive surface and the center of the incident beam is the corresponding reference distance. In order to acquire the second light intensity measurement value and/or the second light intensity reference value, the following manner may be adopted.

For the detected site of the detected object, the incident beam corresponding to each predetermined wavelength is transmitted to the surface of the detected site through the light source entrance. The second light intensity measurement value emitted from the surface of the detected site after each incident beam passes through the detected site is acquired based on the measurement photosensitive surface corresponding to each predetermined wavelength, and/or the second light intensity reference value emitted from the surface of the detected site after each incident beam passes through the detected site is acquired based on the reference photosensitive surface corresponding to each predetermined wavelength.

According to embodiments of the present disclosure, the measurement photosensitive surface and the reference photosensitive surface described above may belong to a linear photosensitive surface array. Accordingly, each measurement photosensitive surface may be understood as an original photosensitive surface whose source-detection distance from the center of the incident beam is the corresponding measurement distance, and each reference photosensitive surface may be understood as an original photosensitive surface whose source-detection distance from the center of the incident beam is the corresponding reference distance. The above-mentioned linear photosensitive surface array may be a diode array detector or may be formed by a linear arrangement of different detectors. The linear photosensitive surface array formed by a linear arrangement of different detectors may be understood as that the linear photosensitive surface array is formed by a linear arrangement of at least two detectors, each detector is independent, and each detector is provided with a corresponding original photosensitive surface.

In addition, the linear photosensitive surface array may be a contact linear photosensitive surface array or a non-contact linear photosensitive surface array. The contact linear photosensitive surface array may be understood as a linear photosensitive surface array in contact with the surface of the detected site, and the non-contact linear photosensitive surface array may be understood as a linear photosensitive surface array in non-contact with the surface of the detected site. Based on the above, the linear photosensitive surface array may be a contact linear photosensitive surface array, which may be a diode array detector or formed by a linear arrangement of different detectors. The linear photosensitive surface array may be a non-contact linear photosensitive surface array, which may be a diode array detector or formed by a linear arrangement of different detectors.

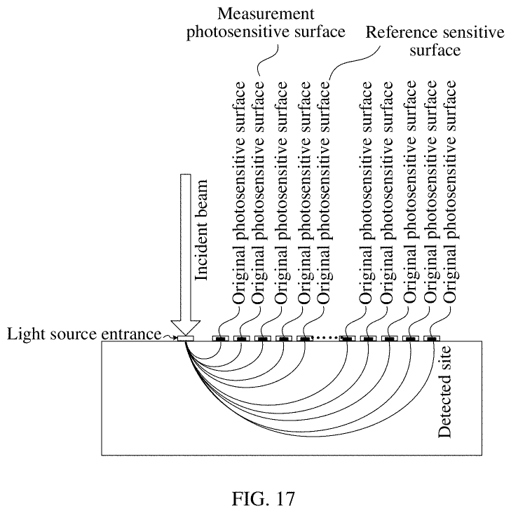

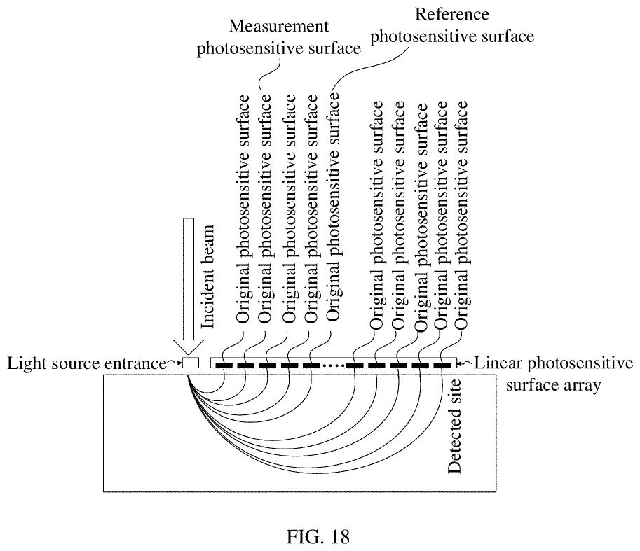

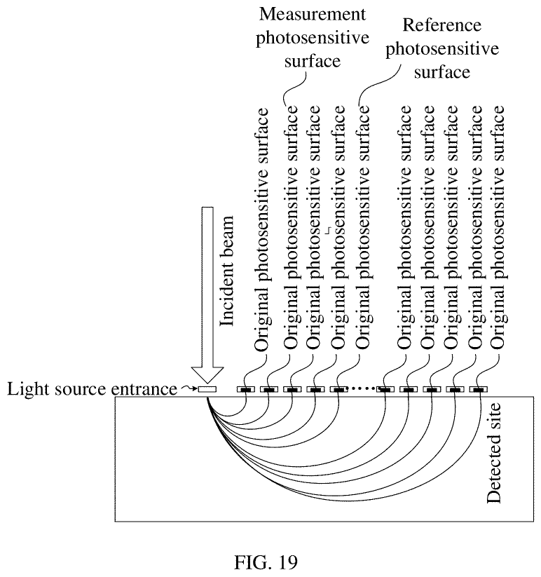

For example, as shown in FIG. 16, FIG. 16 shows a schematic diagram of acquiring the second light intensity measurement value and the second light intensity reference value emitted from the surface of the detected site based on a contact linear photosensitive surface array according to embodiments of the present disclosure. The contact linear photosensitive surface array is a diode array detector. As shown in FIG. 17, FIG. 17 shows another schematic diagram of acquiring the second light intensity measurement value and the second light intensity reference value emitted from the surface of the detected site based on a contact linear photosensitive surface array according to embodiments of the present disclosure, where the contact linear photosensitive surface array is formed by a linear arrangement of different detectors. As shown in FIG. 18, FIG. 18 shows a schematic diagram of acquiring the second light intensity measurement value and the second light intensity reference value emitted from the surface of the detected site based on a non-contact linear photosensitive surface array according to embodiments of the present disclosure. As shown in FIG. 19, FIG. 19 shows another schematic diagram of acquiring the second light intensity measurement value and the second light intensity reference value emitted from the surface of the detected site based on a non-contact linear photosensitive surface array according to embodiments of the present disclosure, where the non-contact linear photosensitive surface array is formed by a linear arrangement of different detectors.

According to embodiments of the present disclosure, the measurement distance and/or the reference distance may be accurately determined, and the second light intensity measurement value and/or the second light intensity reference value may be accurately determined according to the accurately determined measurement distance and/or reference distance, in combination with the reception manner of by the measurement photosensitive surface and/or the reference photosensitive surface. As the concentration of the tissue element to be detected is determined according to the accurately determined second light intensity measurement value and/or the second light intensity reference value, the detection accuracy may be improved.

According to embodiments of the present disclosure, each measurement photosensitive surface and each reference photosensitive surface belong to a linear photosensitive surface array, and the linear photosensitive surface array includes at least two original photosensitive surfaces.

According to embodiments of the present disclosure, each measurement photosensitive surface and each reference photosensitive surface belong to a linear photosensitive surface array, and the linear photosensitive surface array includes at least two original photosensitive surfaces. Each measurement photosensitive surface is an original photosensitive surface whose source-detection distance from the center of the incident beam is a corresponding measurement distance, and each reference photosensitive surface is an original photosensitive surface whose source-detection distance from the center of the incident light beam is a corresponding reference distance.

As shown in FIG. 16 to FIG. 19, according to embodiments of the present disclosure, the linear photosensitive surface array is a diode array detector or is formed by a linear arrangement of different detectors.

According to embodiments of the present disclosure, as shown in FIG. 16 and FIG. 18, the linear photosensitive surface array may be a diode array detector. As shown in FIG. 17 and FIG. 19, the linear photosensitive surface array may be formed by a linear arrangement of different detectors, and each detector is provided with a corresponding original photosensitive surface.

As shown in FIG. 7 to FIG. 8 and FIG. 16 to FIG. 19, according to embodiments of the present disclosure, the light source entrance is in contact or non-contact with the surface of the detected site; and/or the linear photosensitive surface array is in contact or non-contact with the surface of the detected site.

According to embodiments of the present disclosure, the form of the non-invasive detection of tissue element may include a contact detection and a non-contact detection. The contact detection may include the following two manners. In a first manner, the light source entrance is in contact with the surface of the detected site, and/or the linear photosensitive surface array is in contact with the surface of the detected site, as shown in FIG. 16 and FIG. 17. In a second manner, the light source entrance is in contact with the first end of the light guide part array, the linear photosensitive surface array is arranged at the first end of the light guide part array, and the second end of the light guide part array is in contact with the surface of the detected site, as shown in FIG. 7.

The non-contact detection may include the following two manners. In a first manner, the light source entrance and the linear photosensitive surface array are in non-contact with the surface of the detected site, the incident beam from the light source entrance is not transmitted through the light guide part array, and the linear photosensitive surface array receives the second light intensity measurement value and/or the second light intensity reference value that do/does not pass through the light guide part array, as shown in FIG. 18 and FIG. 19. In a second manner, the light source entrance is in contact with the first end of the light guide part array, the linear photosensitive surface array is arranged at the first end of the light guide part array, and the second end of the light guide part array is in non-contact with the surface of the detected site, as shown in FIG. 8. For the description of the contact detection and the non-contact detection, reference may be made to the corresponding parts above, and details are not repeated here.

As shown in FIG. 7 and FIG. 8, according to embodiments of the present disclosure, the non-contact between the light source entrance and the surface of the detected site and the non-contact between the linear photosensitive surface array and the surface of the detected site may be achieved by an arrangement that the light source entrance is in contact with the first end of the light guide part array, the linear photosensitive surface array is arranged at the first end of the light guide part array, the second end of the light guide part array is in contact or non-contact with the surface of the detected site, and the second end of the light guide part array and the first end of the light guide part array are opposite end faces.

According to embodiments of the present disclosure, in order to achieve the non-contact between the light source entrance and the surface of the detected site and the non-contact between the linear photosensitive surface array and the surface of the detected site, the light source entrance may be arranged in contact with the first end of the light guide part array, and the linear photosensitive surface array may be arranged at the first end of the light guide part array. For the description of the arrangement that the light source entrance is in contact with the first end of the light guide part array and the linear photosensitive surface array is arranged at the first end of the light guide part array, reference may be made to the corresponding parts above, and details are not repeated here.

According to embodiments of the present disclosure, the light guide part array includes at least one emission light guide part and a receiving light guide part array. The receiving light guide part array includes at least two receiving light guide parts. A distance between first ends of two adjacent receiving light guide parts is greater than or equal to a distance between second ends of two adjacent receiving light guide parts. A cross-sectional area of the first end of each receiving light guide part is greater than or equal to a cross-sectional area of the second end of each receiving light guide part.

According to embodiments of the present disclosure, for the specific description of the light guide part array, reference may be made to the corresponding parts above, and details are not repeated here.

According to embodiments of the present disclosure, operation 420 may include the following operations. For each predetermined wavelength, a difference operation is performed between the second light intensity measurement value and the second light intensity reference value corresponding to the predetermined wavelength, so as to obtain a light intensity difference value. The concentration of the tissue element to be detected may be determined according to the light intensity difference value corresponding to each predetermined wavelength.

According to embodiments of the present disclosure, in order to further improve the detection accuracy, for each predetermined wavelength, a difference operation is performed between the second light intensity measurement value and the second light intensity reference value corresponding to the predetermined wavelength, so as to obtain the light intensity difference value corresponding to the predetermined wavelength. Based on this, the light intensity difference value corresponding to each predetermined wavelength may be obtained, and the concentration of the tissue element to be detected may be determined according to the light intensity difference value corresponding to each predetermined wavelength. The determining the concentration of the tissue element to be detected according to the light intensity difference value corresponding to each predetermined wavelength may be understood as follows. The light intensity difference value corresponding to each predetermined wavelength may be input into a pre-trained tissue element prediction model to obtain a prediction result, and the prediction result is the concentration of the tissue element to be detected. The specific calculation process may refer to patent document published on Nov. 23, 2005 with a publication number CN1699973A, which will not be described in detail here.

According to embodiments of the present disclosure, the second light intensity reference value corresponding to the reference distance reflects the response caused by the interference other than the change in the concentration of the tissue element to be detected in the detection process, while the second light intensity measurement value corresponding to the measurement distance reflects the response of the tissue element to be detected and the response of the interference other than the tissue element to be detected. Therefore, the reference-measurement may be used, that is, the second light intensity reference value corresponding to the reference distance may be used to correct the second light intensity measurement value corresponding to the measurement distance, so as to eliminate common mode interference to the greatest extent, thereby further improving the detection accuracy.

According to embodiments of the present disclosure, the light source entrance and the linear photosensitive surface array are in non-contact with the surface of the detected site. Before acquiring, based on the measurement photosensitive surface corresponding to each predetermined wavelength, the second light intensity measurement value that is emitted from the surface of the detected site after each incident beam passes through the detected site, the method may further include an operation of shielding interference light.

According to embodiments of the present disclosure, after the incident beam is transmitted to the detected site, a part of the incident beam may be directly reflected on the surface of the detected site to form a surface-reflected light, and a part of the incident beam passes through the detected site and a diffusely-scattered light (i.e., the second light intensity measurement value and the second light intensity reference value) is emitted from the surface of the detected site. The surface-reflected light does not interact with the tissue and therefore may not carry valid information. The valid information may be understood as a response caused by the change in the concentration of the tissue element to be detected in the detection process. Therefore, the surface-reflected light may be regarded as interference light. The diffusely-scattered light interacts with the skin tissue and carries valid information, and thus may be regarded as valid light.

If the light source entrance is in non-contact with the surface of the detected site, the surface-reflected light may be generated. Based on this, in order to further improve the detection accuracy, the interference light may be shielded before acquiring, based on the measurement photosensitive surface corresponding to each predetermined wavelength, the second light intensity measurement value emitted from the surface of the detected site after each incident beam passes through the detected site, so that the second light intensity measurement value emitted from the surface of the detected site after each incident beam passes through the detected site is acquired based on the measurement photosensitive surface corresponding to each predetermined wavelength. The interference light may be shielded in the following two manners.

In a first manner, if the light source entrance and the linear photosensitive surface array are in non-contact with the surface of the detected site, the incident beam from the light source entrance is not transmitted through the light guide part array, and the linear photosensitive surface array receives the second light intensity measurement value and/or the second light intensity reference value that do/does not pass through the light guide part array, then a first light blocking part may be provided in a gap region between the light source entrance and the surface of the detected site, and/or a second light blocking part may be provided in a gap region between the linear photosensitive surface array and the surface of the detected site, as shown in FIG. 10.

In a second manner, if the light source entrance is in contact with the first end of the light guide part array, the linear photosensitive surface array is arranged at the first end of the light guide part array, and the second end of the light guide part array is in non-contact with the surface of the detected site, then a third light blocking part may be provided in a gap region between the emission light guide part and the surface of the detected site, and/or a fourth light blocking part may be provided in a gap region between the receiving light guide part array and the surface of the detected site, as shown in FIG. 11.

According to embodiments of the present disclosure, the interference light is shielded before acquiring the second light intensity measurement value and/or the second light intensity reference value emitted from the surface of the detected site after each incident beam passes through the detected site, so that only the diffusely-scattered light is acquired by the linear photosensitive surface array. As the diffusely-scattered light carries the valid information, the detection accuracy may be further improved.

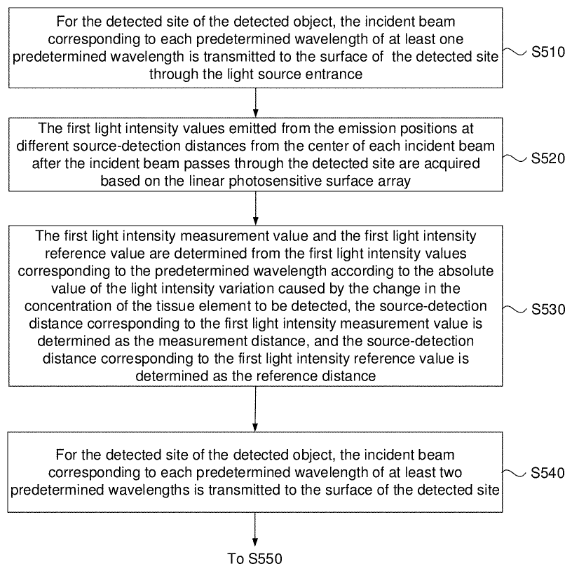

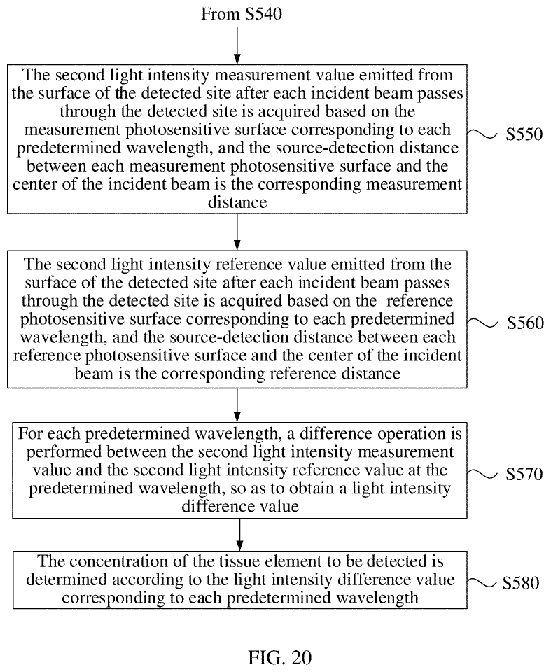

FIG. 20 shows a flowchart of another non-invasive detection method for a tissue element according to embodiments of the present disclosure. Such embodiments may be applied to improve the detection accuracy of the concentration of the tissue element to be detected.

As shown in FIG. 20, the method includes operations S510 to S580.

In operation S510, for the detected site of the detected object, the incident beam corresponding to each predetermined wavelength of at least one predetermined wavelength is transmitted to the surface of the detected site through the light source entrance.

In operation S520, the first light intensity values emitted from the emission positions at different source-detection distances from the center of each incident beam after the incident beam passes through the detected site are acquired based on the linear photosensitive surface array.

According to embodiments of the present disclosure, the linear photosensitive surface array includes at least two original photosensitive surfaces, each original photosensitive surface corresponds to one emission position, and at least two source-detection distances are provided.

In operation S530, the first light intensity measurement value and the first light intensity reference value are determined from the first light intensity values corresponding to the predetermined wavelength according to the absolute value of the light intensity variation caused by the change in the concentration of the tissue element to be detected, the source-detection distance corresponding to the first light intensity measurement value is determined as the measurement distance, and the source-detection distance corresponding to the first light intensity reference value is determined as the reference distance.

According to embodiments of the present disclosure, the first light intensity measurement value is the first light intensity value corresponding to the greatest absolute value of the light intensity variation caused by the change in the concentration of the tissue element to be detected, and the first light intensity reference value is the first light intensity value corresponding to the smallest absolute value of the light intensity variation caused by the change in the concentration of the tissue element to be detected. The light intensity variation caused by the change in the concentration of the tissue element to be detected is the variation between the first light intensity value and the corresponding predetermined light intensity value.

In operation S540, for the detected site of the detected object, the incident beam corresponding to each predetermined wavelength of at least two predetermined wavelengths is transmitted to the surface of the detected site.

In operation S550, the second light intensity measurement value emitted from the surface of the detected site after each incident beam passes through the detected site is acquired based on the measurement photosensitive surface corresponding to each predetermined wavelength. The source-detection distance between each measurement photosensitive surface and the center of the incident beam is the corresponding measurement distance.

In operation S560, the second light intensity reference value emitted from the surface of the detected site after each incident beam passes through the detected site is acquired based on the reference photosensitive surface corresponding to each predetermined wavelength. The source-detection distance between each reference photosensitive surface and the center of the incident beam is the corresponding reference distance.

In operation S570, for each predetermined wavelength, a difference operation is performed between the second light intensity measurement value and the second light intensity reference value at the predetermined wavelength, so as to obtain a light intensity difference value.

In operation S580, the concentration of the tissue element to be detected is determined according to the light intensity difference value corresponding to each predetermined wavelength.

According to embodiments of the present disclosure, each measurement photosensitive surface and each reference photosensitive surface belong to a linear photosensitive surface array, and the linear photosensitive surface array includes at least two original photosensitive surfaces. The light source entrance is in contact or non-contact with the surface of the detected site, and/or the linear photosensitive surface array is in contact or non-contact with the surface of the detected site. The non-contact between the light source entrance and the surface of the detected site and the non-contact between the linear photosensitive surface array and the surface of the detected site may be achieved by an arrangement that the light source entrance is in contact with the first end of the light guide part array, the linear photosensitive surface array is arranged at the first end of the light guide part array, the second end of the light guide part array is in contact or non-contact with the surface of the detected site, and the second end of the light guide part array and the first end of the light guide part array are opposite end faces. If the light source entrance and the linear photosensitive surface array are in non-contact with the surface of the detected site, the method may further include an operation of shielding interference light before operation S520, and an operation of shielding interference light before operation S550. It should be noted that the sequence of execution of operation 550 and operation 560 may be determined according to actual situations, which is not specifically limited here. That is, operation 550 may be performed before operation 560, or operation 560 may be performed before operation 550, or operation 550 and operation 560 may be performed simultaneously.

According to the technical solution of embodiments of the present disclosure, as the first light intensity value corresponding to each predetermined wavelength at each source-detection distance may be acquired, for the detected site of the detected object, based on the linear photosensitive surface array, the first light intensity measurement value and/or the first light intensity reference value may be accurately determined, and the measurement distance and/or the reference distance may be accurately determined. On this basis, the second light intensity measurement value and/or the second light intensity reference value may be accurately determined according to the accurately determined measurement distance and/or reference distance, in combination with the reception manner of the linear photosensitive surface array. As the concentration of the tissue element to be detected is determined according to the accurately determined second light intensity measurement value and/or the second light intensity reference value, the detection accuracy may be improved. Through the difference operation, the common mode interference in the second light intensity reference value and the second light intensity measurement value may be eliminated, so that the detection accuracy may be further improved. In addition, the emission manner of the incident beam and reception manner of the linear photosensitive surface array may greatly reduce the requirements for the photoelectric detector, thereby reducing the manufacturing cost and being easy for implementation.

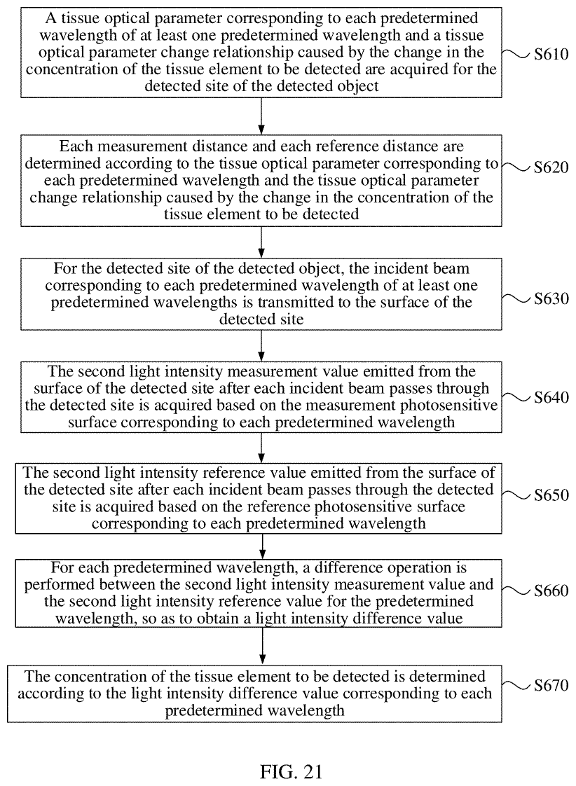

FIG. 21 shows a flowchart of still another non-invasive detection method for a tissue element according to embodiments of the present disclosure. Such embodiments may be applied to improve the detection accuracy of the concentration of the tissue element to be detected.

As shown in FIG. 21, the method includes operations S610 to S670.

In operation S610, a tissue optical parameter corresponding to each predetermined wavelength of at least one predetermined wavelength and a tissue optical parameter change relationship caused by the change in the concentration of the tissue element to be detected are acquired for the detected site of the detected object.

In operation S620, each measurement distance and each reference distance are determined according to the tissue optical parameter corresponding to each predetermined wavelength and the tissue optical parameter change relationship caused by the change in the concentration of the tissue element to be detected.

In operation S630, for the detected site of the detected object, the incident beam corresponding to each predetermined wavelength is transmitted to the surface of the detected site. At least two predetermined wavelengths are provided.

In operation S640, the second light intensity measurement value emitted from the surface of the detected site after each incident beam passes through the detected site is acquired based on the measurement photosensitive surface corresponding to each predetermined wavelength. The source-detection distance between each measurement photosensitive surface and the center of the incident beam is the corresponding measurement distance.

In operation S650, the second light intensity reference value emitted from the surface of the detected site after each incident beam passes through the detected site is acquired based on the reference photosensitive surface corresponding to each predetermined wavelength. The source-detection distance between each reference photosensitive surface and the center of the incident beam is the corresponding reference distance.

In operation S660, for each predetermined wavelength, a difference operation is performed between the second light intensity measurement value and the second light intensity reference value for the predetermined wavelength, so as to obtain a light intensity difference value.

In operation S670, the concentration of the tissue element to be detected is determined according to the light intensity difference value corresponding to each predetermined wavelength.

According to embodiments of the present disclosure, each measurement photosensitive surface and each reference photosensitive surface belong to a linear photosensitive surface array. The light source entrance is in contact or non-contact with the surface of the detected site, and/or the linear photosensitive surface array is in contact or non-contact with the surface of the detected site. The non-contact between the light source entrance and the surface of the detected site and the non-contact between the linear photosensitive surface array and the surface of the detected site may be achieved by an arrangement that the light source entrance is in contact with the first end of the light guide part array, the linear photosensitive surface array is arranged at the first end of the light guide part array, the second end of the light guide part array is in contact or non-contact with the surface of the detected site, and the second end of the light guide part array and the first end of light guide part array are opposite end faces. If the light source entrance and the linear photosensitive surface array are in non-contact with the surface of the detected site, the method may further include an operation of shielding interference light before operation S640. It should be noted that the sequence of execution of operation 640 and operation 650 may be determined according to actual situations, which is not specifically limited here. That is, operation 640 may be performed before operation 650, or operation 650 may be performed before operation 640, or operation 640 and operation 650 may be performed simultaneously.

The method of determining the distance in the non-invasive detection of the tissue element described in embodiments of the present disclosure may be performed by a device of determining a distance in a non-invasive detection of a tissue element, and the non-invasive detection method for the tissue element may be performed by a non-invasive detection device for a tissue element. The device of determining the distance in the non-invasive detection of the tissue element and the non-invasive detection device for the tissue element may be implemented in software and/or hardware, and the non-invasive detection device for the tissue element may be configured in a wearable apparatus, such as a smart watch.

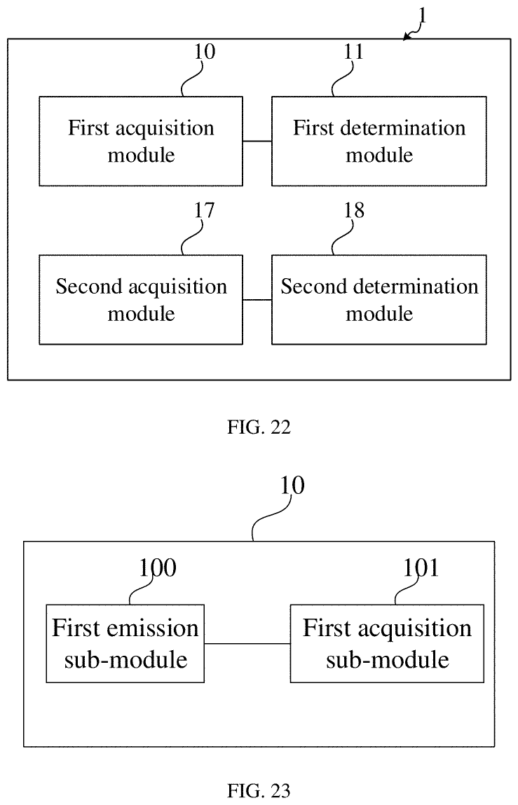

FIG. 22 shows a schematic structural diagram of a device of determining a distance in a non-invasive detection of a tissue element according to embodiments of the present disclosure. Such embodiments may be applied to improve the detection accuracy of the concentration of the tissue element to be detected.

As shown in FIG. 22, a device 1 of determining a distance in a non-invasive detection of a tissue element includes a first acquisition module 10 and a first determination module 11. A structure and an operating principle will be described below with reference to the accompanying drawings.

The first acquisition module 10 is used to acquire a first light intensity value corresponding to each predetermined wavelength of at least one predetermined wavelength at each source-detection distance of at least two source-detection distances for the detected site of the detected object.

The first determination module 11 is used to determine a first light intensity measurement value and/or a first light intensity reference value from the first light intensity values corresponding to the predetermined wavelength according to an absolute value of a light intensity variation caused by the change in the concentration of the tissue element to be detected, determine a source-detection distance corresponding to the first light intensity measurement value as a measurement distance, and determine a source-detection distance corresponding to the first light intensity reference value as a reference distance. The first light intensity measurement value is a first light intensity value corresponding to a greatest absolute value of the light intensity variation caused by the change in the concentration of the tissue element to be detected, the first light intensity reference value is a first light intensity value corresponding to a smallest absolute value of the light intensity variation caused by the change in the concentration of the tissue element to be detected, and the light intensity variation caused by the change in the concentration of the tissue element to be detected is a variation between the first light intensity value and the corresponding predetermined light intensity value.

According to embodiments of the present disclosure, for the specific processing procedures of the first acquisition module 10 and the first determination module 11, reference may be made to the above description in the corresponding part of the method of determining the distance in the non-invasive detection of the tissue element, and details are not repeated here.

As shown in FIG. 23, according to embodiments of the present disclosure, the first acquisition module 10 includes a first emission sub-module 100 and a first acquisition sub-module 101.

The first emission sub-module 100 is used to emit, for the detected site of the detected object, an incident beam corresponding to each predetermined wavelength to a surface of the detected site through the light source entrance.

The first acquisition sub-module 101 is used to acquire, based on the linear photosensitive surface array, the first light intensity values emitted from the emission positions at different source-detection distances from the center of each incident beam after the incident beam passes through the detected site. The linear photosensitive surface array includes at least two original photosensitive surfaces, and each original photosensitive surface corresponds to one emission position.

According to embodiments of the present disclosure, for the specific processing procedures of the first emission sub-module 100 and the first acquisition sub-module 101, reference may be made to the above description in the corresponding part of the method of determining the distance in the non-invasive detection of the tissue element, and details are not repeated here.

As shown in FIG. 3 to FIG. 6, according to embodiments of the present disclosure, the linear photosensitive surface array is a diode array detector or formed by a linear arrangement of different detectors.

As shown in FIG. 3 to FIG. 8, according to embodiments of the present disclosure, the light source entrance is in contact or non-contact with the surface of the detected site, and/or the linear photosensitive surface array is in contact or non-contact with the surface of the detected site.

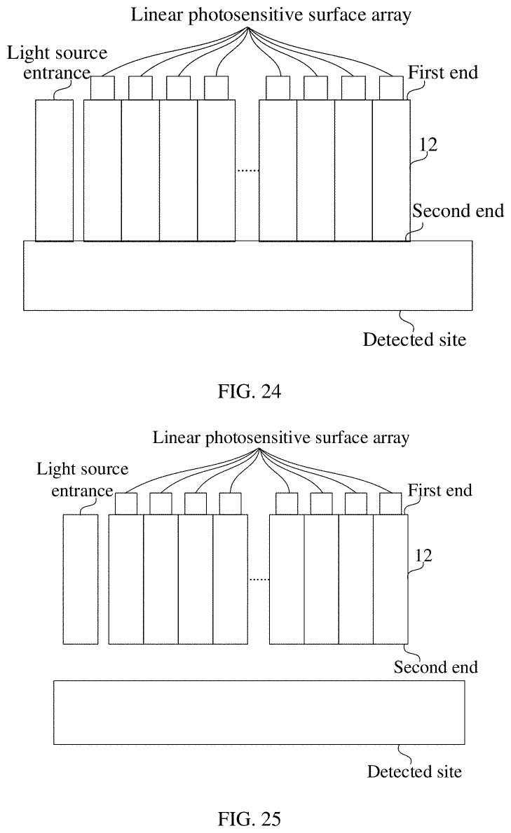

As shown in FIG. 24 and FIG. 25, according to embodiments of the present disclosure, the device 1 of determining the distance in the non-invasive detection of tissue element further includes a light guide part array 12. The light source entrance is in contact with the first end of the light guide part array 12, the linear photosensitive surface array is arranged at the first end of the light guide part array 12, the second end of the light guide part array 12 is in contact or non-contact with the surface of the detected site, and the second end of the light guide part array 12 and the first end of the light guide part array 12 are opposite end faces.

According to embodiments of the present disclosure, in order to achieve the non-contact between the light source entrance and the surface of the detected site and the non-contact between the linear photosensitive surface array and the surface of the detected site, the light source entrance may be arranged in contact with the first end of the light guide part array 12, and the linear photosensitive surface array may be arranged at the first end of the light guide part array 12. As shown in FIG. 24, FIG. 24 shows a schematic diagram of still another linear photosensitive surface array in non-contact with the surface of the detected site according to embodiments of the present disclosure. If the light source entrance is in contact with the first end of the light guide part array 12, the linear photosensitive surface array is arranged at the first end of the light guide part array 12, and the second end of the light guide part array 12 is in non-contact with the surface of the detected site, it may be considered that the non-invasive detection of tissue element is the non-contact detection. As shown in FIG. 25, FIG. 25 shows a schematic diagram of still another linear photosensitive surface array in non-contact with the surface of the detected site according to embodiments of the present disclosure. For the description of the arrangement that the light source entrance is in contact with the first end of the light guide part array and the linear photosensitive surface array is arranged at the first end of the light guide part array 12, reference may be made to the corresponding parts above, and details are not repeated here.

According to embodiments of the present disclosure, the light guide part array 12 includes at least one emission light guide part and a receiving light guide part array. The receiving light guide part array includes at least two receiving light guide parts. A distance between first ends of two adjacent receiving light guide parts is greater than or equal to a distance between second ends of two adjacent receiving light guide parts. A cross-sectional area of the first end of each receiving light guide part is greater than or equal to a cross-sectional area of the second end of each receiving light guide part.

According to embodiments of the present disclosure, in order to improve the detection accuracy, it is necessary to improve a spatial resolution and a light intensity signal-to-noise ratio. The spatial resolution may be improved by setting numerous and dense source-detection distances, and the light intensity signal-to-noise ratio may be improved by selecting a photoelectric detector (i.e., the original photosensitive surface) with a large photosensitive area. In order to meet the requirements of both aspects, the light guide part array may be arranged as a fan-shaped light guide part array, that is, the distance between the first ends of two adjacent receiving light guide parts is greater than the distance between the second ends of two adjacent receiving light guide parts. In this way, the first end of the light guide part array may be provided with a photoelectric detector with a large photosensitive area, and the second end of the light guide part array may be provided with numerous and dense source-detection distances.

According to embodiments of the present disclosure, if a photoelectric detector with a large size is selected to further improve the light intensity signal-to-noise ratio, the end faces of the receiving light guide part may be set to be gradient, that is, each receiving light guide part may be set so that the cross-sectional area of the first end of the receiving light guide part is greater than the cross-sectional area of the second end of the receiving light guide part.

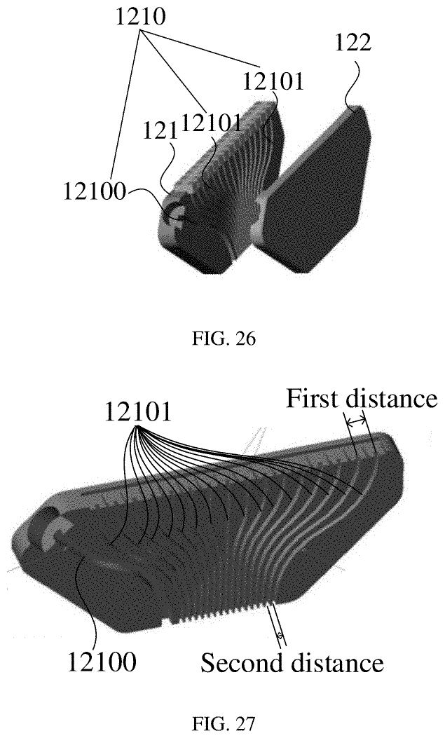

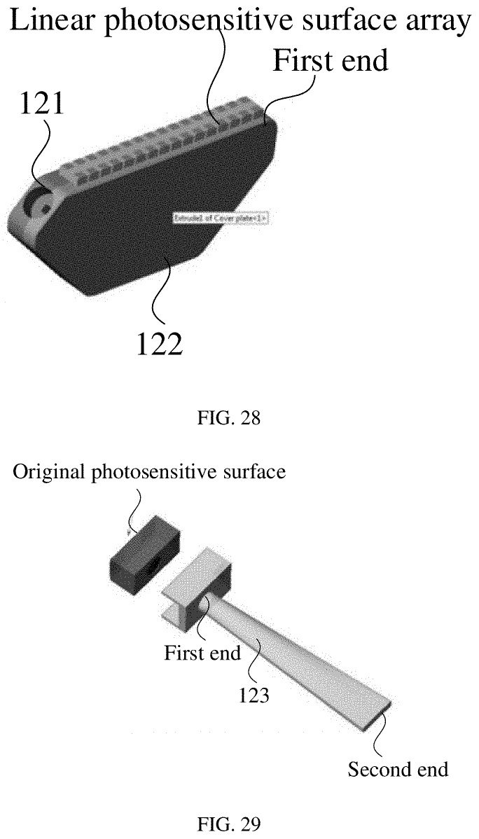

As shown in FIG. 26 to FIG. 28, according to embodiments of the present disclosure, the light guide part array 12 includes a first flat housing 121 and a second flat housing 122. The first flat housing 121 is provided with a light guide groove array 1210, which includes one emission light guide groove 12100 and at least two receiving light guide grooves 12101. The first flat housing 121 may be engaged with the second flat housing 122. After the first flat housing 121 and the second flat housing 122 are engaged, a first end of the first flat housing 121 and a first end of the second flat housing 122 form a groove. The emission light guide part is formed by the emission light guide groove 12100 and the second flat housing 122. Each receiving light guide part is formed by each receiving light guide groove 12101 and the second flat housing 122. The light source entrance is arranged in contact with the first end of the emission light guide groove 12100. The linear photosensitive surface array is embedded in the groove so that each original photosensitive surface is arranged at the first end of the corresponding receiving light guide groove 12100.

According to embodiments of the present disclosure, as shown in FIG. 26 and FIG. 27, the first flat housing 121 may be provided with the light guide groove array 1210, which may include one emission light guide groove 12100 and at least two receiving light guide grooves 12101. The first flat housing 121 may be engaged with the second flat housing 122. After the first flat housing 121 and the second flat housing 122 are engaged, the light guide groove array 1210 may become a hollow light pipe array. A surface of the light guide groove array 1210 provided on the first flat housing 121 is coated with film, and an inner surface of the second flat housing 122 is coated with film. Alternatively, an inner surface of the first flat housing 121 is coated with film, and the inner surface of the second flat housing 122 is coated with film.

After the first flat housing 121 and the second flat housing 122 are engaged, the first end of the first flat housing 121 and the first end of the second flat housing 122 form a groove, and the linear photosensitive surface array may be embedded in the groove. As the linear photosensitive surface array includes at least two original photosensitive surfaces and the light guide groove array 1210 includes at least two receiving light guide grooves 12101, each original photosensitive surface may be arranged at the first end of the corresponding receiving light guide groove 12101. The second end of the receiving light guide groove 12101 may be in contact or non-contact with the surface of the detected site, the light source entrance may be in contact with the first end of the emission light guide groove 12100, and the second end of the emission light guide groove 12100 may be in contact or non-contact with the surface of the detected site.

According to embodiments of the present disclosure, the emission light guide part is formed by the emission light guide groove 12100 and the second flat housing 122, and each receiving light guide part is formed by each receiving light guide groove 12101 and the second flat housing 122. Accordingly, a distance between the first ends of two adjacent receiving light guide grooves 12101 is a distance between the first ends of two adjacent receiving light guide parts, and a distance between the second ends of two adjacent receiving light guide grooves 12101 is a distance between the second ends of two adjacent receiving light guide parts. A cross-sectional area of the first end of each receiving light guide groove 12101 is a cross-sectional area of the first end of each receiving light guide part, and a cross-sectional area of the second end of each receiving light guide groove 12101 is a cross-sectional area of the second end of each receiving light guide part. The distance between the first ends of two adjacent receiving light guide grooves 12101 may be greater than or equal to the distance between the second ends of two adjacent receiving light guide grooves 12101. The cross-sectional area of the first end of each receiving light guide groove 12101 may be greater than or equal to the cross-sectional area of the second end of each receiving light guide groove 12101.

According to embodiments of the present disclosure, if the distance between the first ends of two adjacent receiving light guide grooves 12101 is greater than the distance between the second ends of two adjacent receiving light guide grooves 12101, the light guide groove array 1210 may be a fan-shaped light guide groove array, which may improve the spatial resolution and the light intensity signal-to-noise ratio. The distance between the first ends of two adjacent receiving light guide grooves 12101 may be referred to as a first distance, and the distance between the second ends of two adjacent receiving light guide grooves 12101 may be referred to as a second distance. Accordingly, if the first distance is greater than the second distance, the light guide groove array 1210 may be a fan-shaped light guide groove array, as shown in FIG. 27. In addition, if the cross-sectional area of the first end of each receiving light guide groove 12101 is greater than the cross-sectional area of the second end of each receiving light guide groove 12101, that is, the end surfaces of each receiving light guide groove 12101 are gradient, it is possible to select a photoelectric detector with a larger size to further improve the light intensity signal-to-noise ratio.

According to embodiments of the present disclosure, the surface of the light guide groove array 1210 provided on the first flat housing 121 is coated with film, and the inner surface of the second flat housing 122 is coated with film. Alternatively, the inner surface of the first flat housing 121 is coated with film, and the inner surface of the second flat housing 122 is coated with film.

According to embodiments of the present disclosure, as described above, the hollow light pipe array is adopted, and the method of coating with film causes the incident beam to be reflected and transmitted directly on an inner surface of a cavity, which may avoid an attenuation of a light energy caused by a light reflection at an interface in a case of using other materials, and may reduce an influence of a human-machine interface on the detection result.

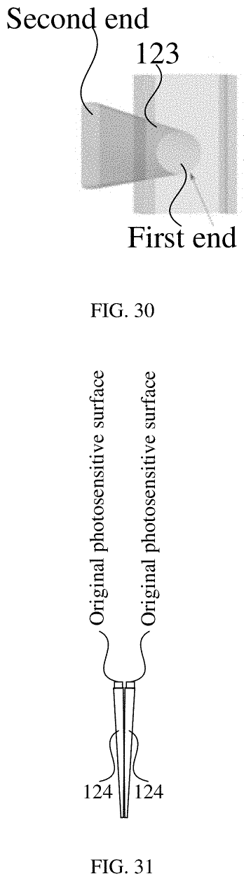

According to embodiments of the present disclosure, the emission light guide part is an emission light guide rod. As shown in FIG. 29 and FIG. 30, each receiving light guide part may be a receiving light guide rod 123. A first end of the emission light guide rod is in contact with the light source entrance, and a first end of each receiving light guide rod 123 is provided with a corresponding original photosensitive surface.

According to embodiments of the present disclosure, as shown in FIG. 29, the first end of each receiving light guide rod 123 may be directly injection-molded with an opening for clamping the corresponding original photosensitive surface, and each original photosensitive surface may be adhered in the opening using a coupling adhesive. The opening may be a rectangular opening. The above may avoid a total internal reflection loss when leaving a plastic medium.

According to embodiments of the present disclosure, the distance between the first ends of two adjacent receiving light guide rods 123 is the distance between the first ends of two adjacent receiving light guide parts, and the distance between the second ends of two adjacent receiving light guide rods 123 is the distance between the second ends of two adjacent receiving light guide parts. A cross-sectional area of the first end of each receiving light guide rod 123 is the cross-sectional area of the first end of each receiving light guide part, and a cross-sectional area of the second end of each receiving light guide rod 123 is the cross-sectional area of the second end of each receiving light guide part. A distance between the first ends of two adjacent receiving light guide rods 123 may be greater than or equal to a distance between the second ends of two adjacent receiving light guide rods 123, and the cross-sectional area of the first end of each receiving light guide rod 123 may be greater than or equal to the cross-sectional area of the second end of each receiving light guide rod 123.

According to embodiments of the present disclosure, if the distance between the first ends of two adjacent receiving light guide rods 123 is greater than the distance between the second ends of two adjacent receiving light guide rods 123, the light guide part array 12 may be a fan-shaped light guide rod array, which may improve the spatial resolution and the light intensity signal-to-noise ratio. In addition, if the cross-sectional area of the first end of each receiving light guide rod 123 is greater than the cross-sectional area of the second end of each receiving light guide rod 123, it is possible to select a photoelectric detector with a larger size to further improve the light intensity signal-to-noise ratio, as shown in FIG. 29 and FIG. 30.

According to embodiments of the present disclosure, an outer surface of the emission light guide rod and an outer surface of each receiving light guide rod 123 are coated with film.

According to embodiments of the present disclosure, as mentioned above, the outer surface of the emission light guide rod and the outer surface of each receiving light guide rod 123 are coated with film, and the receiving light guide rods 123 are not communicated with each other. The above-mentioned arrangement of the receiving light guide rod 123 has no angle limitation of total internal reflection.

According to embodiments of the present disclosure, the emission light guide part is an emission solid light guide sheet. As shown in FIG. 31, each receiving light guide part may be a receiving solid light guide sheet 124. A surface of the emission solid light guide sheet and a surface of each receiving solid light guide sheet 124 are coated with film. A first end of the emission solid light guide sheet is in contact with the light source entrance. A first end of each receiving solid light guide sheet 124 is provided with a corresponding original photosensitive surface.

According to embodiments of the present disclosure, the emission solid light guide sheet and each receiving solid light guide sheet 124 may be bar-shaped transparent plastic or bar-shaped transparent glass, and the surface of the emission solid light guide sheet and the surface of each receiving solid light guide sheet 124 are coated with film. The above-mentioned emission solid light guide sheet may be adhered to at least two receiving solid light guide sheets 124, or the emission solid light guide sheet and at least two receiving solid light guide sheets 124 may be pressed and then the exterior of the emission solid light guide sheet and the receiving solid light guide sheets 124 may be encapsulated with epoxy resin. In addition, the first and second ends of the emission solid light guide sheet and the first and second ends of each receiving solid light guide sheet 124 may be polished. The second end of the emission solid light guide sheet and the second end of the receiving solid light guide sheet 124 may be in contact or non-contact with the surface of the detected site. The emission solid light guide sheet and the receiving solid light guide sheet 124 described above may be used to achieve a high light transmission efficiency and may be flexibly adapted to the arrangement of the original photosensitive surface.

According to embodiments of the present disclosure, a distance between the first ends of two adjacent receiving solid light guide sheets 124 is the distance between the first ends of two adjacent receiving light guide parts, and a distance between the second ends of two adjacent receiving solid light guide sheets 124 is the distance between the second ends of two adjacent receiving light guide parts. A cross-sectional area of the first end of each receiving solid light guide sheet 124 is the cross-sectional area of the first end of each receiving light guide part, and a cross-sectional area of the second end of each receiving solid light guide sheet 124 is the cross-sectional area of the second end of each receiving light guide part. The distance between the first ends of two adjacent receiving solid light guide sheets 124 may be greater than or equal to the distance between the second ends of two adjacent receiving solid light guide sheets 124. The cross-sectional area of the first end of each receiving solid light guide sheet 124 may be greater than or equal to the cross-sectional area of the second end of each receiving solid light guide sheet 124.

According to embodiments of the present disclosure, if the distance between the first ends of two adjacent receiving solid light guide sheets 124 is greater than the distance between the second ends of two adjacent receiving solid light guide sheets 124, the light guide part array 12 may be a fan-shaped solid light guide sheet array, which may improve the spatial resolution and the light intensity signal-to-noise ratio. In addition, if the cross-sectional area of the first end of each receiving solid light guide sheet 124 is greater than the cross-sectional area of the second end of each receiving solid light guide sheet 124, it is possible to select a photoelectric detector with a large size to further improve the light intensity signal-to-noise ratio.

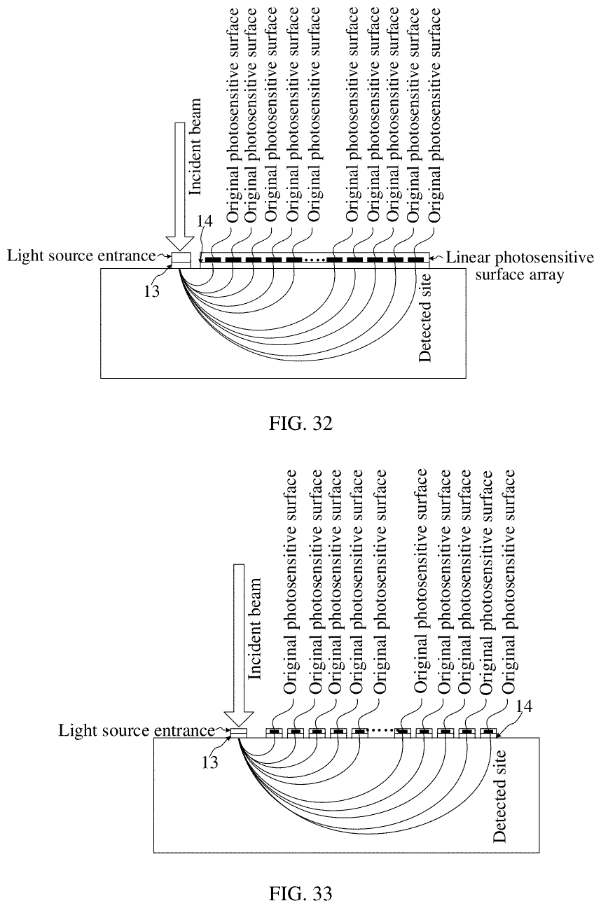

As shown in FIG. 32 and FIG. 33, according to embodiments of the present disclosure, the light source entrance and the linear photosensitive surface array are in non-contact with the surface of the detected site. The device may further include a first light blocking part 13 and/or a second light blocking part 14. The first light blocking part 13 is arranged in a gap region between the light source entrance and the surface of the detected site, and the first light blocking part 13 is in contact with the surface of the detected site. The light source entrance is arranged inside the first light blocking part 13. The first light blocking part 13 is integral with the light source entrance or the first light blocking part 13 is separate from the light source entrance. The second light blocking part 14 is arranged in a gap region between the linear photosensitive surface array and the surface of the detected site, and the second light blocking part 14 is in contact with the surface of the detected site. The linear photosensitive surface array is arranged inside the second light blocking part 14. The second light blocking part 14 is integral with the linear photosensitive surface array or the second light blocking part 14 is separate from the linear photosensitive surface array.

According to embodiments of the present disclosure, if the light source entrance and the linear photosensitive surface array are in non-contact with the surface of the detected site, surface-reflected light may be generated. Based on this, in order to further improve the detection accuracy, the interference light needs to be shielded as follows.

The device 1 of determining the distance in the non-invasive detection of tissue element may be further provided with the first light blocking part 13 and/or the second light blocking part 14. The first light blocking part 13 is specifically arranged in the gap region between the light source entrance and the surface of the detected site, and the first light blocking part 13 is arranged around the light source entrance, so that the light source entrance is located inside the first light blocking part 13, while ensuring that the first light blocking part 13 is in contact with the surface of the detected site; and/or the second light blocking part 14 is specifically arranged in the gap region between the linear photosensitive surface array and the surface of the detected site, and the second light blocking part 14 is arranged around the linear photosensitive surface array, so that the linear photosensitive surface array is located inside the second light blocking part 14, while ensuring that the second light blocking part 14 is in contact with the surface of the detected site. As shown in FIG. 32, another schematic diagram of shielding the interference light is shown. As shown in FIG. 33, still another schematic diagram of shieling the interference light according to embodiments of the present disclosure is shown.

According to embodiments of the present disclosure, the first light blocking part 13 may be integral with or separate from the light source entrance. The second light blocking part 14 may be integral with the linear photosensitive surface array, that is, the second light blocking part 14 may serve as a periphery of the linear photosensitive surface array, which is integral with the linear photosensitive surface array. In addition, the second light blocking part 14 may also be separate from the linear photosensitive surface array. The above may be set according to actual situations, which is not specifically limited here.

According to embodiments of the present disclosure, only the diffusely-scattered light is received by the linear photosensitive surface array. As the diffusely-scattered light carries valid information, the detection accuracy may be further improved.

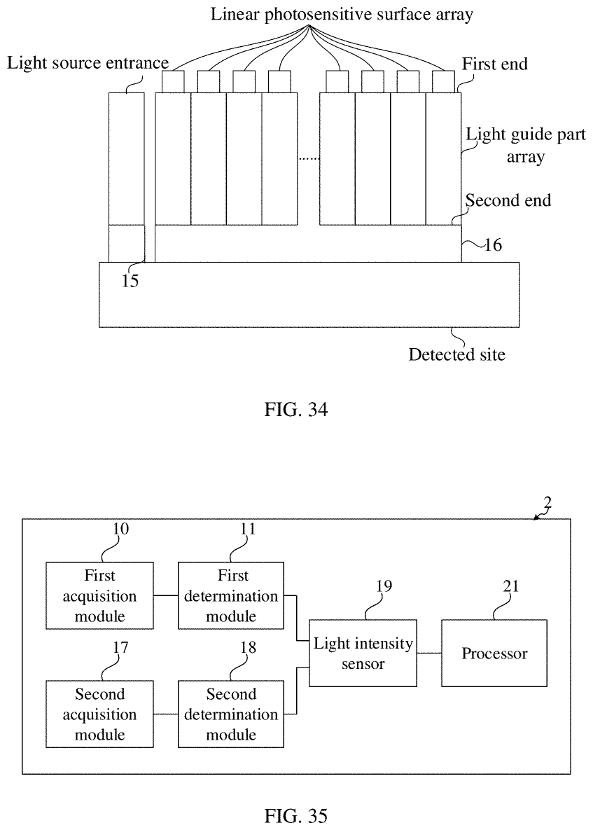

As shown in FIG. 34, according to embodiments of the present disclosure, the second end of the light guide part array 12 is in non-contact with the surface of the detected site. The device 1 of determining the distance in the non-invasive detection of tissue element further includes a third light blocking part 15 and/or a fourth light blocking part 16. The third light blocking part 15 is arranged in a gap region between the emission light guide part and the surface of the detected site, a first end of the third light blocking part 15 is in contact with the second end of the emission light guide part, a second end of the third light blocking part 15 is in contact with the surface of the detected site, and the second end of the third light blocking part 15 and the first end of the third light blocking part 15 are opposite end faces. The fourth light blocking part 16 is arranged in a gap region between the receiving light guide part array and the surface of the detected site, a first end of the fourth light blocking part 16 is in contact with the second end of the receiving light guide part array, a second end of the fourth light blocking part 16 is in contact with the surface of the detected site, and the second end of the fourth light blocking part 16 and the first end of the fourth light blocking part 16 are opposite end faces.

According to embodiments of the present disclosure, if the light source entrance is in contact with the first end of the light guide part array 12, the linear photosensitive surface array is arranged at the first end of the light guide part array 12, and the second end of the light guide part array 12 is in non-contact with the surface of the detected site, it may be considered that the non-invasive detection of tissue element is the non-contact detection. Surface-reflected light may be generated in the non-contact detection. Therefore, in order to further improve the detection accuracy, the interference light needs to be shielded as follows.

The device 1 of determining the distance in the non-invasive detection of tissue element may be further provided with the third light blocking part 15 and/or the fourth light blocking part 16. Specifically, the first end of the third light blocking part 15 is in contact with the second end of the emission light guide part and/or the first end of the fourth light blocking part 16 is in contact with the second end of the receiving light guide part array, and the second end of the third light blocking part 15 and the second end of the fourth light blocking part 16 are in contact with the surface of the detected site, so as to ensure that it is difficult for the interference light to enter the light guide part array 12 and then be received by the linear photosensitive surface array. As shown in FIG. 34. FIG. 34 shows still another schematic diagram of shielding the interference light according to embodiments of the present disclosure.

According to the embodiments of the present disclosure, only the diffusely-scattered light may be received by the linear photosensitive surface array. As the diffusely-scattered light carries valid information, the detection accuracy may be further improved.

FIG. 22 shows a schematic structural diagram of a device of determining a distance in the non-invasive detection of tissue element according to embodiments of the present disclosure. Such embodiments may be applied to improve the detection accuracy of the concentration of the tissue element to be detected.

As shown in FIG. 22, a device 1 of determining a distance in the non-invasive detection of tissue element includes a second acquisition module 17 and a second determination module 18. A structure and an operating principle will be described below with reference to the accompanying drawings.

The second acquisition module 17 is used to acquire, for the detected site of the detected object, the tissue optical parameter at each predetermined wavelength of at least one predetermined wavelength and the tissue optical parameter change relationship caused by the change in the concentration of the tissue element to be detected.

The second determination module 18 is used to determine each measurement distance and/or each reference distance according to the tissue optical parameter at each predetermined wavelength and the tissue optical parameter change relationship caused by the change in the concentration of the tissue element to be detected.

According to embodiments of the present disclosure, for the specific processing procedures of the second acquisition module 17 and the second determination module 18, reference may be made to the description of the corresponding part of the method of determining the distance in the non-invasive detection of tissue element described above, and details are not repeated here.

FIG. 35 shows a schematic structural diagram of a non-invasive detection device for tissue element according to embodiments of the present disclosure. Such embodiments may be applied to improve the detection accuracy of the concentration of the tissue element to be detected.

As shown in FIG. 35, a non-invasive detection device 2 for tissue element includes a light intensity sensor 19 and a processor 21. A structure and an operating principle will be described below with reference to the accompanying drawings.

The light intensity sensor 19 is used to acquire, for the detected site of the detected object, the second light intensity measurement value corresponding to each predetermined wavelength of at least one predetermined wavelength at the measurement distance, and/or the second light intensity reference value corresponding to each predetermined wavelength of at least one predetermined wavelength at the reference distance. Each measurement distance and each reference distance are determined according to the device according to embodiments of the present disclosure.

The processor 21 is used to determine the concentration of the tissue element to be detected according to the second light intensity measurement value and/or the second light intensity reference value corresponding to each predetermined wavelength.

According to embodiments of the present disclosure, for the specific processing procedures of the light intensity sensor 19 and the processor 21, reference may be made to the description of the corresponding part of the non-invasive detection method for tissue element described above, and details are not repeated here.

According to embodiments of the present disclosure, the light intensity sensor 19 includes a beam generator, a first light intensity sensor, and a second light intensity sensor.

The beam generator is used to: for the detected site of the detected object, emit an incident beam corresponding to each predetermined wavelength to the surface of the detected site through the light source entrance.

The first light intensity sensor is used to acquire, based on the measurement photosensitive surface corresponding to each predetermined wavelength, the second light intensity measurement value emitted from the surface of the detected site after each incident beam passes through the detected site. The light-source distance between each measurement photosensitive surface and the center of the incident beam is a corresponding measurement distance.

The second light intensity sensor is used to acquire, based on the reference photosensitive surface corresponding to each predetermined wavelength, the second light intensity reference value emitted from the surface of the detected site after each incident beam passes through the detected site. The light-source distance between each reference photosensitive surface and the center of the incident beam is a corresponding reference distance.

According to embodiments of the present disclosure, for the specific processing procedures of the beam generator, the first light intensity sensor and the second light intensity sensor, reference may be made to the description of the corresponding part of the non-invasive detection method for tissue element described above, and details are not repeated here.

According to embodiments of the present disclosure, each measurement photosensitive surface and each reference photosensitive surface belong to a linear photosensitive surface array, and the linear photosensitive surface array includes at least two original photosensitive surfaces.

As shown in FIG. 16 to FIG. 19, according to embodiments of the present disclosure, the linear photosensitive surface array is a diode array detector or is formed by a linear arrangement of different detectors.

As shown in FIG. 7 to FIG. 8 and FIG. 16 to FIG. 19, according to embodiments of the present disclosure, the light source entrance is in contact or non-contact with the surface of the detected site, and/or the linear photosensitive surface array is in contact or non-contact with the surface of the detected site.

As shown in FIG. 24 and FIG. 25, according to embodiments of the present disclosure, the non-invasive detection device 2 for tissue element further includes a light guide part array 12. The light source entrance is in contact with the first end of the light guide part array 12, the linear photosensitive surface array is provided at the first end of the light guide part array 12, the second end of the light guide part array 12 is in contact or non-contact with the surface of the detected site, and the second end of the light guide part array 12 and the first end of the light guide part array 12 are opposite end faces.

According to embodiments of the present disclosure, the light guide part array 12 includes an emission light guide part and a receiving light guide part array. The receiving light guide part array includes at least two receiving light guide parts. A distance between the first ends of two adjacent receiving light guide parts is greater than or equal to a distance between the second ends of two adjacent receiving light guide parts. A cross-sectional area of the first end of each receiving light guide part is greater than or equal to a cross-sectional area of the second end of each receiving light guide part.

According to embodiments of the present disclosure, for the specific description of the light guide part array 12, reference may be made to the corresponding parts above, and details are not described here.

As shown in FIG. 26 to FIG. 28, according to embodiments of the present disclosure, the light guide part 12 includes a first flat housing 121 and a second flat housing 122. The first flat housing 121 is provided with a light guide groove array 1210, which includes one emission light guide groove 12100 and at least two receiving light guide grooves 12101.

The first flat housing 121 may be engaged with the second flat housing 122. After the first flat housing 121 and the second flat housing 122 are engaged, the first end of the first flat housing 121 and the first end of the second flat housing 122 may form a groove. The emission light guide part is formed by the emission light guide groove 12100 and the second flat housing 122, and each receiving light guide part is formed by each receiving light guide groove 12101 and the second flat housing 122. The light source entrance is arranged in contact with the first end of the emission light guide groove 12100. The linear photosensitive surface array is embedded in the groove so that each original photosensitive surface is arranged at the first end of the corresponding receiving light guide groove 12101.

According to embodiments of the present disclosure, for the specific description of the light guide part array 12, reference may be made to the corresponding parts above, and details are not described here.

According to embodiments of the present disclosure, a surface of the light guide groove array 1210 provided on the first flat housing 121 is coated with film, and an inner surface of the second flat housing 122 is coated with film. Alternatively, an inner surface of the first flat housing 121 is coated with film, and the inner surface of the second flat housing 122 is coated with film.

As shown in FIG. 29 and FIG. 30, according to embodiments of the present disclosure, the emission light guide part is an emission light guide rod, and each receiving light guide part may be a receiving light guide rod 123. The first end of the emission light guide rod is in contact with the light source entrance, and the first end of each receiving light guide rod 123 is provided with a corresponding original photosensitive surface.

According to embodiments of the present disclosure, for the specific description of the receiving light guide rod 123, reference may be made to the corresponding parts above, and details are not described here.

According to embodiments of the present disclosure, an outer surface of the emission light guide rod and an outer surface of each receiving light guide rod 123 are coated with film.

According to embodiments of the present disclosure, the outer surface of the emission light guide rod and the outer surface of each receiving light guide rod 123 are coated with film, and the receiving light guide rods 123 are not communicated with each other. The above-mentioned arrangement of the receiving light guide rod 123 has no angle limitation of total internal reflection.

As shown in FIG. 31, according to embodiments of the present disclosure, the emission light guide part is an emission solid light guide sheet, and each receiving light guide part may be a receiving solid light guide sheet 124. The surface of the emission solid light guide sheet and the surface of each receiving solid light guide sheet 124 are coated with film. The first end of the emission solid light guide sheet is in contact with the light source entrance, and the first end of each receiving solid light guide sheet 124 is provided with a corresponding original photosensitive surface.

According to embodiments of the present disclosure, for the specific description of the receiving solid light guide sheet 124, reference may be made to the corresponding parts above, and details are not described here.

According to embodiments of the present disclosure, the processor 21 is used to, for each predetermined wavelength, perform a difference operation between the second light intensity measurement value and the second light intensity reference value at the predetermined wavelength, so as to obtain a light intensity difference value; and determine the concentration of the tissue element to be detected according to the light intensity difference value at each predetermined wavelength.

According to embodiments of the present disclosure, for the specific processing procedures of the difference sub-module and the determination sub-module, reference may be made to the description of the corresponding part of the non-invasive detection method for tissue element described above, which will not be described in detail here.

As shown in FIG. 32 and FIG. 33, according to embodiments of the present disclosure, the light source entrance and the linear photosensitive surface array are in non-contact with the surface of the detected site. The device may further include a first light blocking part 13 and/or a second light blocking part 14. The first light blocking part 13 is arranged in a gap region between the light source entrance and the surface of the detected site, and the first light blocking part 13 is in contact with the surface of the detected site. The light source entrance is arranged inside the first light blocking part 13. The first light blocking part 13 is integral with the light source entrance or the first light blocking part 13 is separate from the light source entrance. The second light blocking part 14 is arranged in a gap region between the linear photosensitive surface array and the surface of the detected site, and the second light blocking part 14 is in contact with the surface of the detected site. The linear photosensitive surface array is arranged inside the second light blocking part 14. The second light blocking part 14 is integral with the linear photosensitive surface array or the second light blocking part 14 is separate from the linear photosensitive surface array.

According to embodiments of the present disclosure, for the specific description of the first light blocking part 13 and the second light blocking part 14, reference may be made to the corresponding parts above, and details are not repeated here.

As shown in FIG. 34, according to embodiments of the present disclosure, the second end of the light guide part array 12 is in non-contact with the surface of the detected site. The non-invasive detection device 2 for tissue element further includes a third light blocking part 15 and/or a fourth light blocking part 16. The third light blocking part 15 is provided in a gap region between the emission light guide part and the surface of the detected site, the first end of the third light blocking part 15 is in contact with the second end of the emission light guide part, the second end of the third light blocking part 15 is in contact with the surface of the detected site, and the second end of the third light blocking part 15 and the first end of the third light blocking part 15 are opposite end faces. The fourth light blocking part 16 is arranged in a gap region between the receiving light guide part array and the surface of the detected site, the first end of the fourth light blocking part 16 is in contact with the second end of the receiving light guide part array, the second end of the fourth light blocking part 16 is in contact with the surface of the detected site, and the second end of the fourth light blocking part 16 and the first end of the fourth light blocking part 16 are opposite end faces.

According to embodiments of the present disclosure, for the specific description of the third light blocking part 15 and the fourth light blocking part 16, reference may be made to the corresponding parts above, and details are not repeated here.

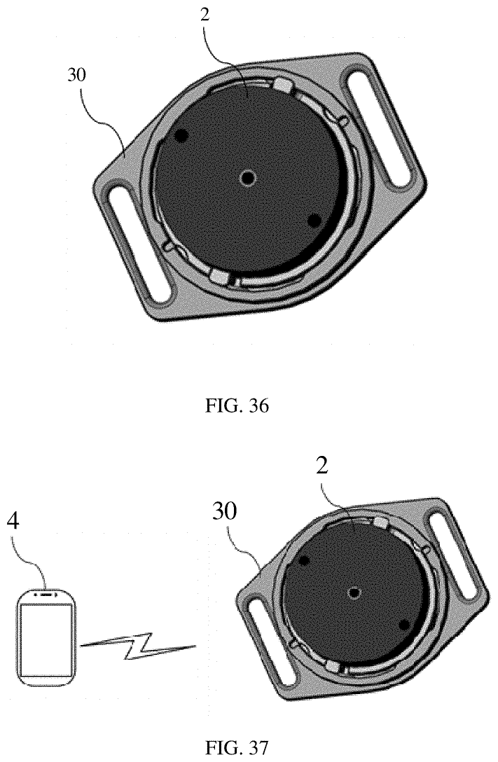

FIG. 36 shows a schematic structural diagram of a wearable apparatus according to embodiments of the present disclosure. Such embodiments may be applied to improve the detection accuracy of the concentration of the tissue element to be detected.

As shown in FIG. 36, the wearable apparatus 3 includes a body 30 and the non-invasive detection device 2 for tissue element according to embodiments of the present disclosure. The non-invasive detection device 2 for tissue element is arranged on the body 30, and the non-invasive detection device 2 for tissue element includes a light intensity sensor 19 and a processor 21. A structure and an operating principle will be described below with reference to the accompanying drawings.

The wearable apparatus 3 is worn on the detected site.

The light intensity sensor 19 is used to, for the detected site of the detected object, acquire the second light intensity measurement value corresponding to each predetermined wavelength of at least one predetermined wavelength at the measurement distance, and/or the second light intensity reference value corresponding to each predetermined wavelength of at least one predetermined wavelength at the reference distance. Each measurement distance and each reference distance are determined according to the device 1 of determining the distance in the non-invasive detection of tissue element described in embodiments of the present disclosure.

The processor 21 is used to determine the concentration of the tissue element to be detected according to the second light intensity measurement value and/or the second light intensity reference value corresponding to each predetermined wavelength.

According to embodiments of the present disclosure, the non-invasive detection device 2 for tissue element may be arranged on the body 30. When the non-invasive detection device 2 for tissue element needs to be used for detection of tissue element, the wearable apparatus 3 may be worn on the detected site. In addition, the detection of the non-invasive detection device 2 for tissue element is easily affected by a detection condition and thus the detection accuracy may be affected. In order to ensure a stability of the detection condition and further improve the detection accuracy, the non-invasive detection device 2 for tissue element may be fixed, so that a positional relationship between the detected site and the non-invasive detection device 2 for tissue element is a predetermined relationship. As described above, the position of the non-invasive detection device 2 for tissue element may be fixed by arranging the non-invasive detection device 2 for tissue element on the body 30, and the stability of the detection condition may be ensured, so that the detection accuracy may be improved. In addition, for the structure and operating principle of the non-invasive detection device 2 for tissue element, reference may be made to the above description for the non-invasive detection device 2, and details are not repeated here.

According to embodiments of the present disclosure, the wearable apparatus 3 may further include a display module communicatively connected with the processor 21. The processor 21 may transmit the concentration of the tissue element to be detected to the display module, and the display module may display the concentration of the tissue element to be detected, so that the concentration of the tissue element to be detected may be obtained by the detected object through the display module. In addition, the wearable apparatus 3 may further include a voice module communicatively connected with the processor 21. The processor 21 may transmit the concentration of the tissue element to be detected to the voice module, and the voice module may generate a voice command according to the concentration of the tissue element to be detected and play the voice command, so that the concentration of the tissue element to be detected may be obtained by the detected object.

In the technical solution of embodiments of the present disclosure, due to a substantial reduction in the size of the detection device, the detection device may be installed on a wearable apparatus, and then easily worn and fixed on the detected site, so that the stability of the detection condition may be ensured, the stability of the detection condition may be improved, and a portable detection may be achieved. On this basis, since the measurement distance and/or the reference distance corresponding to each predetermined wavelength may be accurately determined for the detected site of the detected object, the second light intensity measurement value and/or the second light intensity reference value may be accurately determined according to the accurately determined measurement distance and/or reference distance. The concentration of the tissue element to be detected may be determined according to the accurately determined second light intensity measurement value and/or second light intensity reference value, so that the detection accuracy may be improved.

FIG. 37 shows a schematic structural diagram of a non-invasive detection system for tissue element according to embodiments of the present disclosure. Such embodiments may be applied to improve the detection accuracy of the concentration of the tissue element to be detected.

As shown in FIG. 37, the non-invasive detection system for tissue element includes the wearable apparatus 3 described in embodiments of the present disclosure and a terminal 4. The wearable apparatus 3 includes the body 30 and the non-invasive detection device 2 for tissue element, and the non-invasive detection device 2 for tissue element is arranged on the body 30. The non-invasive detection device 2 for tissue element includes a light intensity sensor 19 and a processor 21. The processor 21 may be communicatively connected with the light intensity sensor 19 and the terminal 4, respectively. A structure and operating principle thereof will be described below with reference to the accompanying drawings.

The wearable apparatus 3 is worn on the detected site.

The light intensity sensor 19 is used to, for the detected site of the detected object, acquire the second light intensity measurement value corresponding to each predetermined wavelength of at least one predetermined wavelength at the measurement distance, and/or the second light intensity reference value corresponding to each predetermined wavelength at the reference distance. Each measurement distance and each reference distance are determined according to the device described in embodiments of the present disclosure.

The processor 21 is used to process each second light intensity measurement value and/or each second light intensity reference value for each predetermined wavelength, so as to obtain each processed second light intensity measurement value and/or each processed second light intensity reference value for each predetermined wavelength, and transmit each processed second light intensity measurement value and/or each processed second light intensity reference value for each predetermined wavelength to the terminal 4.

The terminal 4 is used to determine the concentration of the tissue element to be detected according to each processed second light intensity measurement value and/or each processed second light intensity reference value for each predetermined wavelength.

According to embodiments of the present disclosure, different from preceding embodiments, the wearable apparatus 3 and the terminal 4 may cooperate to determine the concentration of the tissue element to be detected in order to reduce the cost of the non-invasive detection device 2 for tissue element. That is, the processor 21 may process each second light intensity measurement value and/or each second light intensity reference value for each predetermined wavelength to obtain each processed second light intensity measurement value and/or each processed second light intensity reference value for each predetermined wavelength, and transmit each processed second light intensity measurement value and/or each processed second light intensity reference value for each predetermined wavelength to the terminal 4. The terminal 4 may determine the concentration of the tissue element to be detected according to each processed second light intensity measurement value and/or each processed second light intensity reference value for each predetermined wavelength. The processing operation of each second light intensity measurement value and/or each second light intensity reference value by the processor 21 may include current-to-voltage conversion, amplification and analog-to-digital conversion, etc. The terminal 4 may determine the concentration of the tissue element to be detected according to each processed second light intensity measurement value and/or each processed second light intensity reference value by using the same methods as the non-invasive detection methods for tissue element described in embodiments of the present disclosure, which will not be described in detail here. In addition, a structure and an operating principle of the wearable apparatus 3 may be referred to the description of the wearable apparatus 3 above, and details are not repeated here.

According to embodiments of the present disclosure, the terminal 4 may further display the concentration of the tissue element to be detected, so that the concentration of the tissue element to be detected may be obtained by the detected object. The terminal 4 may further generate a voice command containing the concentration of the tissue element to be detected, and play the voice command, so that the concentration of the tissue element to be detected may be obtained by the detected object.

According to embodiments of the present disclosure, in addition to determining the concentration of the tissue element to be detected by the cooperation of the terminal 4 and the wearable apparatus 3, the concentration of the tissue element to be detected may also be determined by a cooperation of a cloud server and the wearable apparatus 3.

The specific embodiments of the present disclosure described above do not limit the protection scope of the present disclosure. Any other corresponding changes and modifications made according to the technical concept of the present disclosure should be included within the protection scope of the claims of the present disclosure.

1. (canceled)

2. (canceled)

3. (canceled)

4. (canceled)

5. (canceled)

6. (canceled)

7. (canceled)

8. (canceled)

9. A non-invasive detection method for a tissue element, comprising:
acquiring, for a detected site of a detected object, a second light intensity measurement value for each predetermined wavelength of at least one predetermined wavelength at a measurement distance, and/or a second light intensity reference value for each predetermined wavelength of at least one predetermined wavelength at a reference distance, wherein the measurement distance is a source-detection distance corresponding to a first light intensity measurement value, and the reference distance is a source-detection distance corresponding to a first light intensity reference value; and
determining a concentration of a tissue element to be detected according to the second light intensity measurement value for each predetermined wavelength and/or the second light intensity reference value for each predetermined wavelength;
wherein the acquiring, for a detected site of a detected object, a second light intensity measurement value for each predetermined wavelength at a measurement distance, and/or a second light intensity reference value for each predetermined wavelength at a reference distance comprises:
emitting, for the detected site of the detected object, an incident beam corresponding to each predetermined wavelength to a surface of the detected site through a light source entrance; and
acquiring, based on a measurement photosensitive surface corresponding to each predetermined wavelength, the second light intensity measurement value emitted from the surface of the detected site after each incident beam passes through the detected site, wherein a source-detection distance between each measurement photosensitive surface and a center of the incident beam is a corresponding measurement distance; and/or
acquiring, based on a reference surface photosensitive surface to each predetermined wavelength, the second light intensity reference value emitted from the surface of the detected site after each incident beam passes through the detected site, wherein a source-detection distance between each reference photosensitive surface and a center of the incident beam is a corresponding reference distance;
wherein each measurement, photosensitive surface and each reference photosensitive surface belong to a linear photosensitive surface array.

10. (canceled)

11. (canceled)

12. (canceled)

13. (canceled)

14. The method according to claim 9, wherein the non-contact between the light source entrance and the surface of the detected site and the non-contact between the linear photosensitive surface array and the surface of the detected site are achieved by:
causing the light source entrance to be in contact with a first end of a light guide part array, the linear photosensitive surface array to be arranged at the first end of the light guide part array, and a second end of the light guide part array to be in contact or non-contact with the surface of the detected site, wherein the second end of the light guide part array and the first end of the light guide part array are opposite end faces.

15. The method according to claim 14, wherein the light guide part array comprises an emission light guide part and a receiving light guide part array, and the receiving light guide part array comprises at least two receiving light guide parts, and
wherein a distance between first ends of two adjacent receiving light guide parts of the at least two receiving light guide parts is greater than or equal to a distance between second ends of two adjacent receiving light guide parts of the at least two receiving light guide parts; and
a cross-sectional area of the first end of each of the at least two receiving light guide parts is greater than or equal to a cross-sectional area of the second end of each of the at least two receiving light guide parts.

16. The method according to claim 9, wherein the determining a concentration of a tissue element to be detected according to the second light intensity measurement value for each predetermined wavelength and/or the second light intensity reference value for each predetermined wavelength comprises:
performing, for each predetermined wavelength, a difference operation between the second light intensity measurement value for the predetermined wavelength and the second light intensity reference value for the predetermined wavelength, so as to obtain a light intensity difference value; and
determining the concentration of the tissue element to be detected according to the light intensity difference value for each predetermined wavelength.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. A non-invasive detection device for a tissue element, comprising:
a light intensity sensor configured to acquire, for a detected site of a detected object, a second light intensity measurement value for each predetermined wavelength of at least one predetermined wavelength at a measurement distance, and/or a second light intensity reference value for each predetermined wavelength of at least one predetermined wavelength at a reference distance, wherein the measurement distance is a source-detection distance corresponding to a first light intensity measurement value, and the reference distance is a source-detection distance corresponding to a first light intensity reference value; and
a processor configured to determine a concentration of a tissue element to be detected according to the second light intensity measurement value for each predetermined wavelength and/or the second light intensity reference value for each predetermined wavelength;
wherein the light intensity sensor comprises:
a beam generator configured to emit, for the detected site of the detected object, an incident beam corresponding to each predetermined wavelength to a surface of the detected site through a light source entrance;
a first light intensity sensor configured to acquire, based on a measurement photosensitive surface corresponding to each predetermined wavelength, the second light intensity measurement value emitted from the surface of the detected site after each incident beam passes through the detected site, wherein a source-detection distance between each measurement photosensitive surface and a center of the incident beam is a corresponding measurement distance; and/or
a second light intensity sensor configured to acquire, based on a reference photosensitive surface corresponding to each predetermined wavelength, the second light intensity reference value emitted from the surface of the detected site after each incident beam passes through the detected site, wherein a source-detection distance between each reference photosensitive surface and a center of the incident beam is a corresponding reference distance;
wherein each measurement photosensitive surface and each reference photosensitive surface belong to a linear photosensitive surface array, and the linear, photosensitive surface array comprises at least two original photosensitive surfaces.

33. (canceled)

34. (canceled)

35. (canceled)

36. (canceled)

37. The device according to claim 32, further comprising a light guide part array,
wherein the light source entrance is in contact with a first end of the light guide part array, the linear photosensitive surface array is arranged at the first end of the light guide part array, a second end of the light guide part array is in contact or non-contact with the surface of the detected site, and the second end of the light guide part array and the first end of the light guide part array are opposite end faces.

38. The device according to claim 37, wherein the light guide part array comprises an emission light guide part and a receiving light guide part array, and the receiving light guide part array comprises at least two receiving light guide parts, and
wherein a distance between first ends of two adjacent receiving light guide parts of the at least two receiving light guide parts is greater than or equal to a distance between second ends of two adjacent receiving light guide parts of the at least two receiving light guide parts; and
a cross-sectional area of the first end of each of the at least two receiving light guide parts is greater than or equal to a cross-sectional area of the second end of each of the at least two receiving light guide parts.

39. The device according to claim 38, wherein the light guide part array comprises a first flat housing and a second flat housing, the first flat housing is provided with a light guide groove array, and the light guide groove array comprises an emission light guide groove and at least two receiving light guide grooves, and
wherein the first flat housing are engaged with the second flat housing, and after the first flat housing and the second flat housing are engaged, a first end of the first flat housing and a first end of the second flat housing form a groove, the emission light guide part is formed by the emission light guide groove and the second flat housing, and each of the at least two receiving light guide parts is formed by each of the at least two receiving light guide grooves and the second flat housing; and
the light source entrance is in contact with a first end of the emission light guide groove, and the linear photosensitive surface array is embedded in the groove so that each of the at least two original photosensitive surfaces is arranged at a first end of a corresponding receiving light guide groove of the at least two receiving light guide grooves.

40. The device according to claim 39, wherein a surface of the light guide groove array provided on the first flat housing is coated with film, and an inner surface of the second flat housing is coated with film; or an inner surface of the first flat housing is coated with film, and the inner surface of the second flat housing is coated with film.

41. The device according to claim 38, wherein the emission light guide part is an emission light guide rod, each of the at least two receiving light guide parts is a receiving light guide rod, and wherein a first end of the emission light guide rod is in contact with the light source entrance, and a first end of the receiving light guide rod is provided with a corresponding original photosensitive surface of the at least two original photosensitive surfaces.

42. (canceled)

43. The device according to claim 38, wherein the emission light guide part is an emission solid light guide sheet, each of the at least two receiving light guide parts is a receiving solid light guide sheet, and
wherein a surface of the emission solid light guide sheet and a surface of the receiving solid light guide sheet are coated with film; and
a first end of the emission solid light guide sheet is in contact with the light source entrance, and a first end of the receiving solid light guide sheet is provided with a corresponding original photosensitive surface of the at least two original photosensitive surfaces.

44. The device according to claim 32, wherein the processor is configured to perform, for each predetermined wavelength, a difference operation on the second light intensity measurement value and the second light intensity reference value for the predetermined wavelength, so as to obtain a light intensity difference value; and
determine the concentration of the tissue element to be detected according to the light intensity difference value for each predetermined wavelength.

45. The device according to claim 32, wherein the light source entrance and the linear photosensitive surface array are in non-contact with the surface of the detected site, and the device further comprises a first light blocking part and/or a second light blocking part, and
wherein the first light blocking part is arranged in a gap region between the light source entrance and the surface of the detected site, the first light blocking part is in contact with the surface of the detected site, the light source entrance is arranged inside the first light blocking part, and the first light blocking part is integral with the light source entrance or the first light blocking part is separate from the light source entrance; and
the second light blocking part is arranged in a gap region between the linear photosensitive surface array and the surface of the detected site, the second light blocking part is in contact with the surface of the detected site, the linear photosensitive surface array is arranged inside the second light blocking part, and the second light blocking part is integral with the linear photosensitive surface array or the second light blocking part is separate from the linear photosensitive surface array.

46. The device according to claim 38, wherein the second end of the light guide part array is in non-contact with the surface of the detected site, and the device further comprises a third light blocking part and/or a fourth light blocking part, and
wherein the third light blocking part is arranged in a gap region between the emission light guide part and the surface of the detected site, a first end of the third light blocking part is in contact with the second end of the emission light guide part, a second end of the third light blocking part is in contact with the surface of the detected site, and the second end of the third light blocking part and the first end of the third light blocking part are opposite end faces; and
the fourth light blocking part is arranged in a gap region between the receiving light guide part array and the surface of the detected site, a first end of the fourth light blocking part is in contact with the second end of the receiving light guide part array, a second end of the fourth light blocking part is in contact with the surface of the detected site, and the second end of the fourth light blocking part and the first end of the fourth light blocking part are opposite end faces.

47. A wearable apparatus, comprising a body and the non-invasive detection device for the tissue element according to claim 32,
wherein the non-invasive detection device for the tissue element is arranged on the body, and the wearable apparatus is worn on the detected site.

48. (canceled)
